(19) United States
(12) Patent Application Publication
(10) Pub. No.: US 2015/0202157 A1
(43) Pub. Date: Jul. 23, 2015

(54) COMPOSITIONS AND METHODS FOR ENHANCING NEURONAL PHOSPHORYLATION HOMEOSTASIS, AND MODULATING DYSFUNCTIONAL EXOCYTOSIS AND NEUROTRANSMITTER RELEASE

(71) Applicant: Revalesio Corporation, Tacoma, WA (US)

(72) Inventors: Richard L. Watson, Ruston, WA (US); Anthony B. Wood, Tacoma, WA (US); Gregory J. Archambeau, Puyallup, WA (US); Supurna Ghosh, Sammamish, WA (US)

(21) Appl. No.: 14/603,305

(22) Filed: Jan. 22, 2015

Related U.S. Application Data
(60) Provisional application No. 61/930,388, filed on Jan. 22, 2014.

(51) Int. Cl. A61K 9/16; A61K 33/14

(52) U.S. Cl. CPC A61K 9/16; A61K 33/14

(57) ABSTRACT

Provided are methods for treating pre-neuronal loss abnormalities in synaptic function, comprising administrating to a subject having neurons, an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies. Also provided are methods for treating pre-neuronal loss abnormalities in synaptic function, comprising contacting neurons in vitro or ex vivo with an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation.

### CROSS-REFERENCE TO RELATED APPLICATIONS

This application claims the benefit of priority to U.S. Provisional Patent Application No. 61/930,388, filed Jan. 22, 2014, and entitled “COMPOSITIONS AND METHODS FOR OPTIMIZING NEURONAL SYNAPTIC TRANSMISSION,” which is incorporated herein by reference in its entirety.

### FEDERAL FUNDING ACKNOWLEDGEMENT

This work was at least in part funded by NIH Grant No. AG027476, and funding from NS/NINDS/NIH HHS. The United States government therefore has certain rights in the invention.

### FIELD OF THE INVENTION

Particular aspects relate generally to neurons and neuronal synaptic transmission, and more particularly to compositions and methods for enhancing neuronal phosphorylation homeostasis, and modulating dysfunctional exocytosis and neurotransmitter release.

### BACKGROUND OF THE INVENTION

Determining the biological variables that control both electrical and chemical synaptic transmission between nerve cells, or between nerve terminals and muscular or glandular systems, has been a very significant area of physiological exploration over the decades. Chemical synaptic transmission has had the added attraction of addressing both the transmission gain of the event, as well as the excitatory or inhibitory nature of the junction and its activity-dependent potentiation or depression.

Taupathies, a class of degenerative human CNS pathologies represent a large category of neurological and psychiatric conditions. Amongst them are Alzheimer's disease, progressive supranuclear palsy, frontotemporal dementia and Pick's disease.

From a neuropathological perspective, ultrastructural analysis indicates intracellular accumulation and aggregation of abnormal filaments, mostly microtubular associated protein tau. corticobasal degeneration, and frontotemporal dementia with Parkinsonism linked to chromosome 17 (FTDP-17) (Lee V M, et al., Neurodegenerative tauopathies. Annu Rev Neurosci 24:1121-1159, 2001). Among these, Alzheimer's disease (AD), demonstrates, in addition filamentous structures, paired helical filaments (PHFs) and straight filaments (SFs). These filaments eventually form large aggregations, known as neurofibrillary tangles (NFTs). Also present in AD are diffuse senile plaques, composed of amyloid beta (Aβ) peptides.

The association between tau filaments, neuron loss, and brain dysfunction in vertebrates and invertebrates originally led to the hypothesis that NFTs invariably cause brain dysfunction and neurodegeneration. However, mouse tauopathy studies indicate that severe abnormalities in synaptic function can precede neuronal loss and even NFTs formation (LaFerla F M, et al., Intracellular amyloid-beta in Alzheimer's disease. Nat Rev Neurosci 8(7):499-509, 2007; and Yoshiyama Y, et al., Synapse loss and microglial activation precede tangles in a P301S tauopathy mouse model. Neuron 53(3):337-351, 2007). The molecular mechanisms responsible for this early malfunction (as well as those responsible for tau polymerization dependent pathogenesis) remain unknown (Marx J, Alzheimer's disease. A new take on tau. Science 316(5830):1416-1417, 2007). Neurofibrillary degeneration is accompanied by lysosomal hypertrophy (Nixon R A, et al., The lysosomal system in neurons. Involvement at multiple stages of Alzheimer's disease pathogenesis. Ann N Y Acad Sci 674:65-88, 1992), beading and degeneration of distal dendrites (Marx J, supra; and Braak E, et al., A sequence of cytoskeleton changes related to the formation of neurofibrillary tangles and neuropil threads. Acta Neuropathol 87(6):554-567, 1994) and axonal damage (Kowall N W & Kosik K S, Axonal disruption and aberrant localization of tau protein characterize the neuropil pathology of Alzheimer's disease. Ann Neurol 22(5):639-643, 1987).

RNS60 is a physically modified normal saline (0.9%) solution generated by using a rotor/stator device, which incorporates controlled turbulence and Taylor-Couette-Poiseuille (TCP) flow under high oxygen pressure (see Applicants U.S. Pat. Nos. 7,832,920; 7,919,534; 8,410,182; 8,445,546; 8,449,172; and 8,470,893, all incorporated herein by reference in their entireties for their teachings encompassing Applicants' device, methods for making the fluids, and the fluids per se).

### SUMMARY OF THE INVENTION

According to particular exemplary aspects, RNS60, a physically modified saline containing charge-stabilized oxygen-containing nanostructures (e.g., charge-stabilized oxygen-containing nanobubbles), has significant function-optimizing properties for enhancing neuronal phosphorylation homeostasis, and modulating dysfunctional exocytosis and neurotransmitter release.

The present study tests whether synaptic optimization via RNS60 ASW could also modify transmission following presynaptic human TAU41 injection.

According to particular aspects RNS60 ASW reduced and even prevented synaptic transmitter block following presynaptic spike activation within fifteen to thirty minutes superfussion. Similar results were obtained concerning spontaneous transmitter release as determined by postsynaptic noise analysis. Thus, TAU injection dependent postsynaptic noise reduction at the post-synaptic axon, concomitant with synaptic block, is also reversed by RNS60 superfusion. This effect occurs without modification of voltage dependent presynaptic calcium current amplitude or kinetics, as demonstrated by presynaptic voltage clamp results.

According to certain aspects, physiological concentrations of recombinant human tau isoform (full length h-tau42) (11) was directly injected into the presynaptic terminal of the squid giant synapse, to examine possible acute effects of h-tau on the synaptic release mechanism. The results showed that heavy exogenous h-tau41 accumulation induces a rapid and short-lasting increase in spontaneous transmitter release followed by a drastic decrease and failure of synaptic transmission. This synaptic block does not affect presynaptic calcium current flow or spike generation at the presynaptic terminal. Immunohistochemistry, performed in h-tau41 injected synapses, demonstrated that h-tau42 becomes phosphorylated rapidly in good temporal agreement with the time course of the transmitter failure (Moreno, H., et al., Front Synaptic Neurosci. 2011; 3: 3, 2011; doi: 10.3389/fnsyn.2011.00003). By contrast immunohistochemistry obtain with the same procedure, but followed by RNS60 ASW prevents such hyperphosphorylation. Electron microscopy and electrophysiological experiments unambiguously indicate that h-tau42-mediated synaptic transmission block is due to exocytosis failure.

The present set of studies identifies several mechanisms of tau-mediated toxicity at the presynaptic terminal, and introduces a potential disease modifier for AD and other tauopathies, and particularly for treating and/or ameliorating and/or preventing severe abnormalities in synaptic function that precede neuronal loss and even NFTs formation, for which there is no specific treatment presently.

Particular aspects provide methods for treating pre-neuronal loss abnormalities in synaptic function, comprising administrating to a subject having neurons, an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies. Additional aspects provide methods for treating pre-neuronal loss abnormalities in synaptic function, comprising contacting neurons in vitro or ex vivo with an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation.

### BRIEF DESCRIPTION OF THE DRAWINGS

FIG. 1 shows, according to particular exemplary aspects, fluorescence imaging of presynaptic TAU 42 injection into the presynatic terminal. The time course for the diffusion of the protein is indicated by the diffusional speed of the fluorescence in the injected preterminal.

FIGS. 2A and 2B show, according to particular exemplary aspects, an increase in latency and final block of postsynaptic spike generation and decrease in amplitude of the postsynaptic potential (lower arrows) at increasing time intervals following TAU injection (FIG. 2A); and that maintenance of synaptic transmission is present after 50 minutes following TAU injection (FIG. 2B, see fluorescence image insert to the right).

FIGS. 3A and 3B show, according to particular exemplary aspects, spontaneous synaptic noise reduction following TAU 41 injection.

FIGS. 4A-4C show, according to particular exemplary aspects: a set of three superimposed voltage clamp results where presynaptic voltage steps (Pre V) that generated presynaptic inward calcium current (I Ca) and postsynaptic potentials (EPSP) are illustrated 10 minutes following presynaptic TAU 41 injection (FIG. 4A); a similar set of voltage clamp results 10 minutes following superfusion with RNS60 based ASW h-tau42 injection (FIG. 4B (upper right quadrant); and before (green) and after (purple) presynaptic to show in more detail the increase on amplitude of the postsynaptic response to the presynaptic voltage steps (FIG. 4C (lower three sets of postsynaptic potentials). The middle traces are the before and after recordings of post synaptic potentials. In the center, is shown a direct comparison of these postsynaptic responses with the difference shown in yellow shading.

FIGS. 5A-5C show, according to particular exemplary aspects, ultrastructural presynaptic changes secondary to h-tau42 injection.

### DETAILED DESCRIPTION

According to particular exemplary aspects, RNS60, a physically modified saline containing charge-stabilized oxygen-containing nanostructures (e.g., charge-stabilized oxygen-containing nanobubbles), has significant function-optimizing properties for enhancing neuronal phosphorylation homeostasis, and modulating dysfunctional exocytosis and neurotransmitter release.

According to particular aspects, RNS60 represents a class of bioactive agents relating to the physical structure of water and an increased oxygen carrying ability (in the form of charge-stabilized oxygen-containing nanostructures, e.g., charge-stabilized oxygen-containing nanobubbles), with no added chemical molecules and yet has proven cytoprotective and anti-inflammatory effects in different models of neurodegeneration through direct effects on glial cells as well as modulation of T cell subsets (Khasnavis 5.2012; Mondal, S, 2012). Without being bound by mechanism, and together with the results described herein, this suggests that RNS60 exerts pleiotropic effects that are not based on interaction with a specific receptor, but rather that RNS60 is a facilitator of physiological function that require a different appellative. Functionally, RNS60 is able to optimize synaptic transmission without affecting normal function and without any deleterious side effects, as has been demonstrated in previous studies in other systems including human use.

### Preferred Embodiments

Particular aspects provide methods for treating pre-neuronal loss abnormalities in synaptic function, comprising administrating to a subject having neurons, an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies. In certain aspects, preventing or reducing abnormalities in synaptic function that precede neuronal loss and/or NFTs formation comprises optimizing phosphorylation homeostasis in the neurons. In particular aspects, optimizing phosphorylation homeostasis in the neurons comprises decreasing the phosphorylated/dephosphorylated ratio in proteins involved in synaptic vesicle function. In certain embodiments, decreasing the phosphorylated/dephosphorylated ratio in proteins involved in synaptic vesicle function comprises modulating tau-induced changes in the balance of kinases and phosphatases in the neurons. In particular aspects, decreasing the phosphorylated/dephosphorylated ratio in proteins involved in synaptic vesicle function comprises decreasing the phosphorylated/dephosphorylated ratio of tau and/or synapsin 1.

In certain aspects of the methods disclosed herein, preventing or reducing abnormalities in synaptic function comprises modulating at least one presynaptic and/or postsynaptic response. In particular aspects, modulating at least one presynaptic and/or postsynaptic response comprises an increase of spontaneous transmitter release. In particular aspects, modulating at least one presynaptic and/or postsynaptic response comprises a modification of noise kinetics. In particular aspects, modulating at least one presynaptic and/or postsynaptic response comprises an increase in a postsynaptic response. In particular aspects, the methods comprise increasing the postsynaptic response without an increase in presynaptic ICa⁺⁺ amplitude. In particular aspects, modulating at least one presynaptic and/or postsynaptic response comprises a decrease in synaptic vesicle density and/or number at active zones. Certain embodiments further comprise an increase in the number of clathrin-coated vesicles, and large endosome like vesicles in the vicinity of the junctional sites. In certain aspects, modulating at least one presynaptic and/or postsynaptic response comprises a marked increase in ATP synthesis leading to synaptic transmission optimization. In certain aspects, modulating at least one presynaptic and/or postsynaptic response comprises an enhanced or more vigorous recovery of postsynaptic spike generation. In certain aspects, modulating at least one presynaptic and/or postsynaptic response comprises increased ATP synthesis at the presynaptic and postsynaptic terminals.

Additional aspects provide methods for treating pre-neuronal loss abnormalities in synaptic function, comprising contacting neurons in vitro or ex vivo with an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies. In certain aspects, preventing or reducing abnormalities in synaptic function that precede neuronal loss and/or NFTs formation comprises optimizing phosphorylation homeostasis in the neurons. In certain aspects, optimizing phosphorylation homeostasis in the neurons comprises decreasing the phosphorylated/dephosphorylated ratio in proteins involved in synaptic vesicle function. In particular aspects, decreasing the phosphorylated/dephosphorylated ratio in proteins involved in synaptic vesicle function comprises modulating tau-induced changes in the balance of kinases and phosphatases in the neurons. In certain aspects, decreasing the phosphorylated/dephosphorylated ratio in proteins involved in synaptic vesicle function comprises decreasing the phosphorylated/dephosphorylated ratio of tau and/or synapsin 1.

Particular aspects provide a method for optimizing neurotransmission, comprising contacting neurons with, or administrating to a subject having neurons, an electrokinetically altered ionic aqueous solution comprising charge-stabilized oxygen-containing nanostructures having an average diameter of less than 100 nm in an amount and for a time period sufficient for modulating at least one presynaptic and/or postsynaptic response, wherein a method for optimizing neuronal synaptic transmission is afforded. In certain aspects, modulating at least one presynaptic and/or postsynaptic response comprises an increase of spontaneous transmitter release. In certain aspects, modulating at least one presynaptic and/or postsynaptic response comprises a modification of noise kinetics. In certain aspects, modulating at least one presynaptic and/or postsynaptic response comprises an increase in a postsynaptic response (e.g., without an increase in presynaptic ICa⁺⁺ amplitude). In certain aspects, modulating at least one presynaptic and/or postsynaptic response comprises a decrease in synaptic vesicle density and/or number at active zones, and may further comprise an increase in the number of clathrin-coated vesicles, and large endosome like vesicles in the vicinity of the junctional sites. In certain aspects, modulating at least one presynaptic and/or postsynaptic response comprises a marked increase in ATP synthesis leading to synaptic transmission optimization. In certain aspects, modulating at least one presynaptic and/or postsynaptic response comprises an enhanced or more vigorous recovery of postsynaptic spike generation. In certain aspects, modulating at least one presynaptic and/or postsynaptic response comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

Additional aspect provide a method for optimizing neurotransmission, comprising contacting neurons with, or administrating to a subject having neurons, an electrokinetically altered ionic aqueous solution comprising charge-stabilized oxygen-containing nanostructures having an average diameter of less than 100 nm in an amount and for a time period sufficient for enhancing intracellular oxygen delivery or utilization, wherein a method for optimizing neuronal synaptic transmission is afforded. In certain aspects, optimizing neuronal synaptic transmission comprises an increase of spontaneous transmitter release. in certain aspects, optimizing neuronal synaptic transmission comprises a modification of noise kinetics. In certain aspects, optimizing neuronal synaptic transmission comprises an increase in a postsynaptic response (e.g., without an increase in presynaptic ICa⁺⁺ amplitude). In certain aspects, optimizing neuronal synaptic transmission comprises a decrease in synaptic vesicle density and/or number at active zones, and may further comprise an increase in the number of clathrin-coated vesicles, and large endosome like vesicles in the vicinity of the junctional sites. In certain aspects, optimizing neuronal synaptic transmission comprises a marked increase in ATP synthesis. In certain aspects, optimizing neuronal synaptic transmission comprises an enhanced or more vigorous recovery of postsynaptic spike generation. in certain aspects, optimizing neuronal synaptic transmission comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

Further aspect provide a method for enhancing intracellular oxygen delivery or utilization, comprising contacting cells with, or administrating to a subject having cells, an electrokinetically altered ionic aqueous solution comprising charge-stabilized oxygen-containing nanostructures having an average diameter of less than 100 nm in an amount and for a time period sufficient for enhancing intracellular oxygen delivery or utilization in the cells. In particular aspects, the cells are nerve cells (e.g., mammalian, human or other; any organism or animal comprising neurons and neuronal transmission). In particular aspects, enhancing intracellular oxygen delivery or utilization provides for optimizing neuronal synaptic transmission. In particular aspects, optimizing neuronal synaptic transmission comprises an increase of spontaneous transmitter release. In particular aspects, optimizing neuronal synaptic transmission comprises a modification of noise kinetics. In particular aspects, optimizing neuronal synaptic transmission comprises an increase in a postsynaptic response (e.g., without an increase in presynaptic ICa⁺⁺ amplitude). In particular aspects, optimizing neuronal synaptic transmission comprises a decrease in synaptic vesicle density and/or number at active zones. In particular aspects, and may further comprise an increase in the number of clathrin-coated vesicles, and large endosome like vesicles in the vicinity of the junctional sites. In particular aspects, optimizing neuronal synaptic transmission comprises a marked increase in ATP synthesis. In particular aspects, optimizing neuronal synaptic transmission comprises an enhanced or more vigorous recovery of postsynaptic spike generation. In particular aspects, optimizing neuronal synaptic transmission comprises increased ATP synthesis at the presynaptic and postsynaptic terminals. In particular embodiments the charge-stabilized oxygen-containing nanostructures having an average diameter of less than 100 nm comprise charge-stabilized oxygen-containing nanobubbles having an average diameter of less than 100 nm.

Consistent with the above, while the underlying pathogenic mechanisms of tau related neuronal abnormalities remain obscure, the acute effects of intra-axonal h-tau42 injection presented here suggest that synaptic dysfunction is an early mechanism in AD and other tauopathies. We used the art-recognized squid giant synapse model in this study because it provides unique advantages in addressing the cellular and molecular mechanisms involved in chemical synaptic transmission. In this set of experiments we determined that h-tau42 produces a rapid failure in exocytosis. Our results also indicate that h-tau42 has previously unknown physiological properties that may be relevant in tau related neurodegenerative process.

According to particular aspects, human tau-42 acutely blocks chemical synaptic transmission without affecting the presynaptic calcium currents or the endocytic pathway.

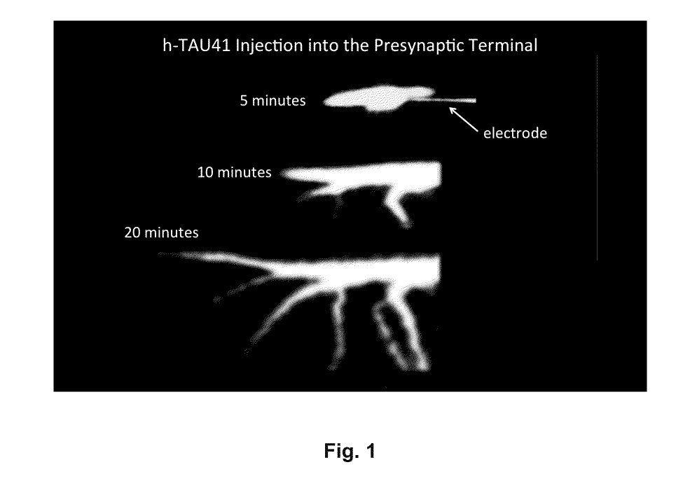

The data presented here indicate that an excess of h-tau42 protein produces synaptic transmission block by interfering with a mechanism of synaptic vesicle exocytosis. Our conclusion, that h-tau42 induces a failure in neurotransmitter availability due to reduced synaptic vesicle release, is derived from morphological, high frequency stimulation, and spontaneous neurotransmitter release data. Moreover, all the h-tau-42 injected synapses demonstrated a drastic block of both spontaneous and evoked transmitted release, without affecting presynaptic spike generation (FIG. 1A) or the associated calcium current. We interpret these findings as reflecting the reduced vesicle count at the active zone, the vesicles being instead concentrated in groups away from the active zone. It was also noted that these electron dense vesicular congregations were characterized by profiles resembling vesicular adhesions to microfilaments as would be expected if synapsin 1 were to be dephosphorylated affording a strong adhesion to such microfilaments (Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985). As a result, h-tau42 would lead to the failure in exocytosis due to both a defect in the release mechanism and a reduction in vesicular availability (Llinas R, et al., Regulation by synapsin I and Ca(2+)-calmodulin-dependent protein kinase II of the transmitter release in squid giant synapse. J Physiol 436:257-282, 1991).

According to particular aspects, human Tau is phosphorylated in the isolated presynaptic terminal and induces abnormal vesicular clustering. Previous experiments in Drosophila have shown that misexpression of human-tau (h-tau), the same isoform as the one used in our experiments, produced significant neurodegeneration (Jackson G R, et al., Humn wild-type tau interacts with wingless pathway components and produces neurofibrillary pathology in Drosophila. Neuron 34(4):509-519, 2002; Avila J, et al., Role of tau protein in both physiological and pathological conditions. Physiol Rev 84(2):361-384, 2004; and Steinhilb M L, et al., Tau phosphorylation sites work in concert to promote neurotoxicity in vivo. Mol Biol Cell 18(12):5060-5068, 2007). In the Drosophila model, tau co-expressed with Shaggy, which generated a single fly homolog of GSK-3β, the phenotype was aggravated (Jackson, supra). Dysfunctional phenotypes were also found in the central neurons of lamprey, where long-term expression (2-38 days) of several h-tau isoforms produced neurodegenerative changes as a result of accumulation of h-hyperphosphorylated tau which correlated with the appearance of structures that resemble AD characteristic—“straight like filaments” (Hall G F, et al., Human tau becomes phosphorylated and forms filamentous deposits when overexpressed in lamprey central neurons in situ. Proc Natl Acad Sci USA 94(9):4733-473, 1997). In the latter experiments the isoform hyperphosphorylated moiety was, to a larger extent, the long form of h-tau (h-tau42) (Hall, supra; and Lee S, et al., Exonic point mutations of human tau enhance its toxicity and cause characteristic changes in neuronal morphology, tau distribution and tau phosphorylation in the lamprey cellular model of tauopathy. J Alzheimers Dis 16(1):99-111, 2009). It has been proposed that the physiological function of tau is adversely affected by excess phosphorylation resulting in tau being displaced from microtubules and aggregating, which in turn leads to microtubule disassembly, disruption of axonal transport, and finally synaptic failure.

Concerning cephalopods, it has been demonstrated that h-tau binds to the squid axonal microtubules, but monomeric h-tau did not affect fast axonal transport (FAT), while filamentous h-tau42 did block anterograde FAT (LaPointe N E, et al., The amino terminus of tau inhibits kinesin-dependent axonal transport: implications for filament toxicity. J Neurosci Res 87(2):440-451, 2009). However, other studies have found that flies misexpressing tau show defects in neuronal traffic without evidence of tau aggregation (Jackson, supra). Finally, extracellular applied h-tau42 to cell cultures produced aberrant signaling through muscarinic receptor activation (Diaz-Hernandez M, et al., Tissue-nonspecific alkaline phosphatase promotes the neurotoxicity effect of extracellular tau. J Biol Chem 285(42):32539-32548, 2010; and Gomez-Ramos A, et al., Extracellular tau promotes intracellular calcium increase through M1 and M3 muscarinic receptors in neuronal cells. Mol Cell Neurosci 37(4):673-681, 2008), suggesting that even ‘normal’ tau may be detrimental when its expression becomes elevated or when it accumulates extracellularly.

According to particular aspects, therefore, it appears that an optimal level of tau phosphorylation is required to achieve the balance in the level of ‘free’ and ‘microtubule bound’ tau that is essential in maintaining microtubule dynamics and subsequent axonal transport.

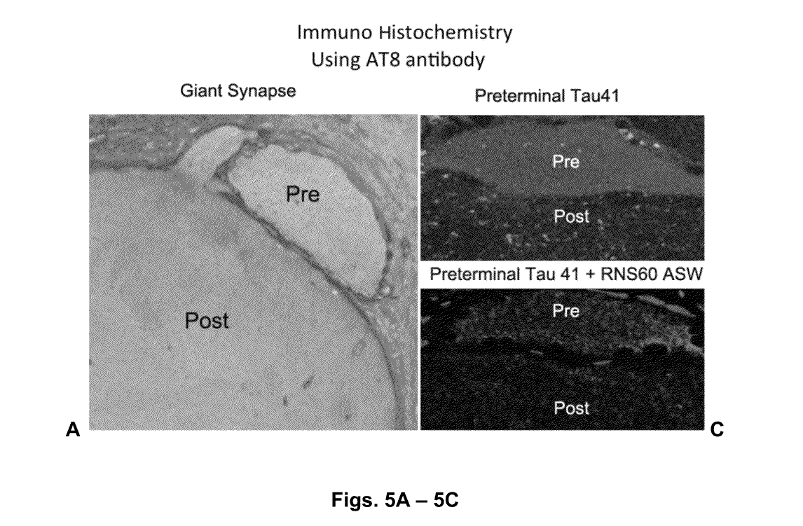

As in the experiments mentioned above, in our experiments, h-tau42 also became phosphorylated in the isolated axon (separated from the cell body) as demonstrated by using AT8 antibodies immunohistochemistry (FIG. 5 B). AT8 recognizes epitopes phosphorylated by GSK3 and cdk5 kinases both of which are found in squid axoplasm (34, 35), suggesting that either one or both kinases may be involved in the effects of h-tau42 in the presynaptic terminal. Whichever the specific kinase, the results demonstrate that isolated axons have the complete machinery to produce local post translational modifications and that these changes may explain, in part, the detrimental effects of excessive “normal tau” on the function of the presynaptic terminal.

Moreover, the vesicle clustering observed in h-tau42 injected synapses, resembled the effect of unphosphorylated synapsin 1 on synaptic vesicle (Jackson, supra). The fact that h-tau42 is phosphorylated intra-axonally and that unphosphorylated synapsin 1 restrains the vesicle pool to the cytoskeleton—producing a decreased number of vesicles available for exocytosis was actually demonstrated some time ago (Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985).

According to particular aspects, therefore, h-tau42 induces changes in the balance of kinases and phosphatases, perhaps influenced by the concentration of h-tau aggregates, thereby decreasing the phosphorylated/dephosphorylated ratio in proteins involved in synaptic vesicle function, such as synapsin 1, which would result in a reduction in the available vesicles and ultimately synaptic transmission failure. Tau is phosphorylated by several protein kinases and this is balanced by protein phosphatases dephosphorylation. The potential kinases and phosphatases involved have been reviewed by Hanger D P, et al., Mediators of tau phosphorylation in the pathogenesis of Alzheimer's disease. Expert Rev Neurother. (11):1647-66, 2009. According to additional aspects, when this process also involves constitutive vesicular dynamics, a secondary dying-back event (Moreno H, et al., Synaptic transmission block by presynaptic injection of oligomeric amyloid beta. Proc Natl Acad Sci USA 106(14):5901-5906, 2009) results in the synaptic disconnection encountered in, for example, AD pathomorphology.

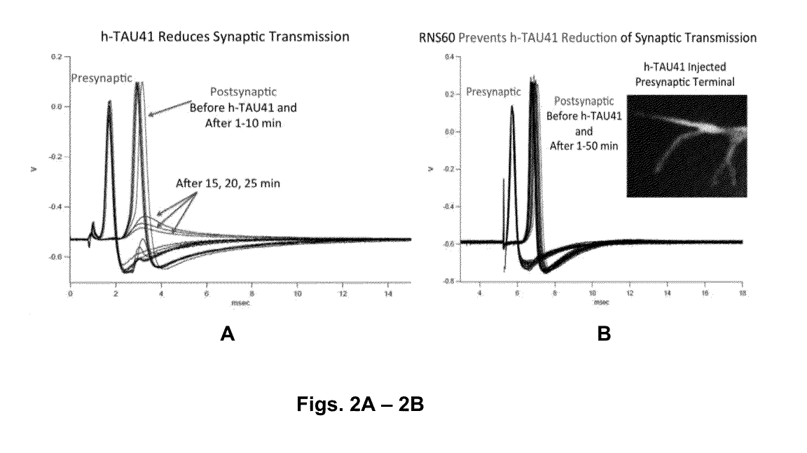

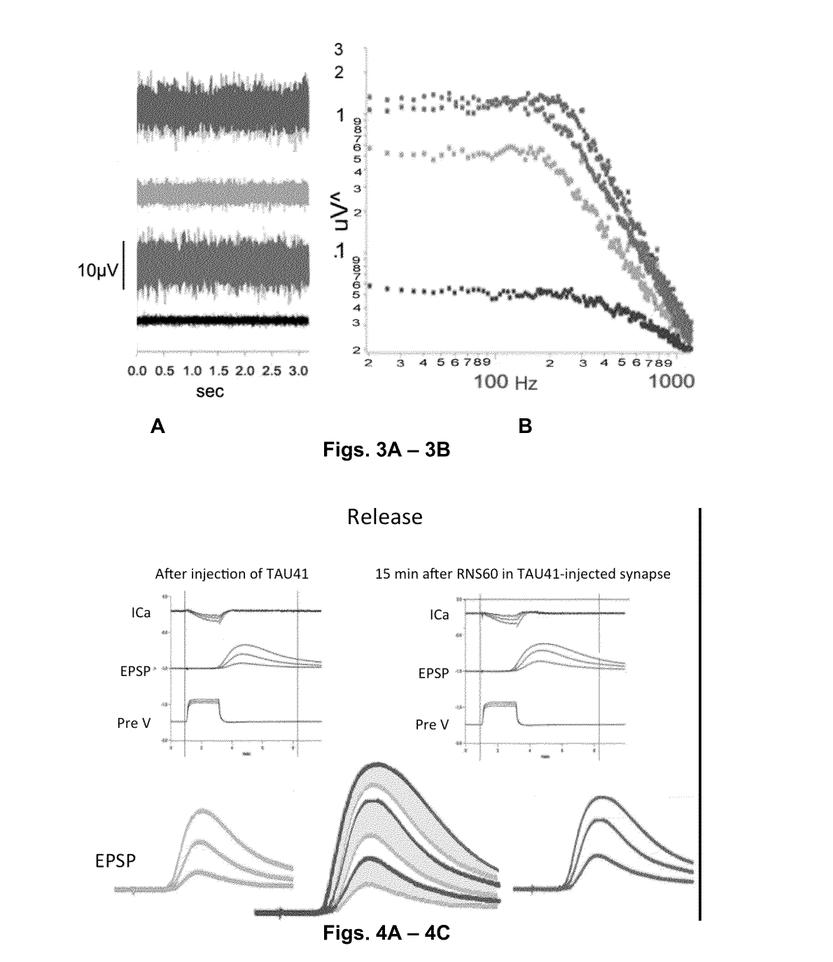

Relating to pharmacological targets of tau mediated neuropathogenesis, according to particular aspects, the present results identify the protective effect of RNS60 superfusion on h-tau42 mediated axonal/synaptic dysfunction as shown in FIG. 2B and FIG. 3. This finding is highly significant, as it has been demonstrated that reduction of endogenous tau in an AD mouse model, ameliorates amyloid beta induced neurodegeneration at several levels (Roberson, E. D., et al., Reducing endogenous tau ameliorates amyloid beta-induced deficits in an Alzheimer's disease mouse model. Science 316, 750-754, 2007). Therefore, according to particular aspects, RNS60 has utility to treat tau pathology. Addressing the precise mechanisms of RNS60 neuroprotection in relation to tau pathology remains to be elucidated. Nonetheless, the fact that both functional and biochemical h-tau42 induced abnormalities in the presynaptic axon are prevented/ameliorated by RNS60 provides a new therapeutic to treat tauopathies.

Our results indicate that hTau-42, affects synaptic release by modifying intracellular phosphorylation dynamics as a resultant of the hTau-42 hyperphosphorylation. This change in the phosphorylation homeostasis results in alteration of the normal intracellular phosphorylation profile leading to a marked reduction of synaptic vesicle availability, most probably due to the reduction of synapsin 1 phosphorylation, known to be a powerful modulator of synaptic release (Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985). Beyond the effecting synaptic release the reduction of such vesicular fusion on constitutive vesicular dynamics would also result in a disconnection event ultimately generating a “dying-back” event (Serulle Y., et al., 1-Methyl-4-phenylpyridinium induces synaptic dysfunction through a pathway involving caspase and PKC∂ enzymatic activities. PNAS 104:2437-2441, 2007; and Pigino G., et al., 1-Methyl-4-phenylpyridinium affects fast axonal transport by activation of caspase and protein kinase C. PNAS 104:2442-2447, 2007).

### Electrokinetically-Generated Fluids:

“Electrokinetically generated fluid,” as used herein, refers to Applicants' inventive electrokinetically-generated fluids generated, for purposes of the working Examples herein, by the exemplary Mixing Device described in detail in Applicants' issued patents (see, e.g., Applicants' issued U.S. Pat. Nos. 7,832,920; 7,919,534; 8,410,182; 8,445,546; 8,449,172; and 8,470,893, all incorporated herein by reference in their respective entireties). The electrokinetic fluids, as demonstrated by the data disclosed and presented herein, represent novel and fundamentally distinct fluids relative to prior art non-electrokinetic fluids, including relative to prior art oxygenated non-electrokinetic fluids (e.g., pressure pot oxygenated fluids and the like). As disclosed in various aspects herein, the electrokinetically-generated fluids have unique and novel physical and biological properties including, but not limited to the following:

In particular aspects, the electrokinetically altered aqueous fluid comprise an ionic aqueous solution of charge-stabilized oxygen-containing nanostructures substantially having an average diameter of less than about 100 nanometers and stably configured in the ionic aqueous fluid in an amount sufficient to provide, upon contact of a living cell by the fluid, modulation of at least one of cellular membrane potential and cellular membrane conductivity.

In particular aspects, electrokinetically-generated fluids refers to fluids generated in the presence of hydrodynamically-induced, localized (e.g., non-uniform with respect to the overall fluid volume) electrokinetic effects (e.g., voltage/current pulses), such as device feature-localized effects as described herein. In particular aspects said hydrodynamically-induced, localized electrokinetic effects are in combination with surface-related double layer and/or streaming current effects as disclosed and discussed herein.

In particular aspects the administered inventive electrokinetically altered fluids comprise charge-stabilized oxygen-containing nanostructures in an amount sufficient to provide modulation of at least one of cellular membrane potential and cellular membrane conductivity. In certain embodiments, the electrokinetically altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically altered fluids are not-superoxygenated (e.g., RNS-10 or Solas, comprising 10 ppm (e.g., approx. ambient levels of dissolved oxygen in standard saline)). In certain aspects, the salinity, sterility, pH, etc., of the inventive electrokinetically altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible.

In particular aspects, the inventive electrokinetically altered fluids comprise saline (e.g., one or more dissolved salt(s); e.g., alkali metal based salts (Li+, Na+, K+, Rb+, Cs+, etc.), alkaline earth based salts (e.g., Mg++, Ca++), etc., or transition metal-based positive ions (e.g., Cr, Fe, Co, Ni, Cu, Zn, etc.,), in each case along with any suitable anion components, including, but not limited to F−, Cl−, Br−, I−, PO4−, SO4−, and nitrogen-based anions. Particular aspects comprise mixed salt based electrokinetic fluids (e.g., Na+, K+, Ca++, Mg++, transition metal ion(s), etc.) in various combinations and concentrations, and optionally with mixtures of couterions. In particular aspects, the inventive electrokinetically altered fluids comprise standard saline (e.g., approx. 0.9% NaCl, or about 0.15 M NaCl). In particular aspects, the inventive electrokinetically altered fluids comprise saline at a concentration of at least 0.0002 M, at least 0.0003 M, at least 0.001 M, at least 0.005 M, at least 0.01 M, at least 0.015 M, at least 0.1 M, at least 0.15 M, or at least 0.2 M. In particular aspects, the conductivity of the inventive electrokinetically altered fluids is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In particular aspects, any salt may be used in preparing the inventive electrokinetically altered fluids, provided that they allow for formation of biologically active salt-stabilized nanostructures (e.g., salt-stabilized oxygen-containing nanostructures) as disclosed herein.

According to particular aspects, the biological effects of the inventive fluid compositions comprising charge-stabilized gas-containing nanostructures can be modulated (e.g., increased, decreased, tuned, etc.) by altering the ionic components of the fluids, and/or by altering the gas component of the fluid.

According to particular aspects, the biological effects of the inventive fluid compositions comprising charge-stabilized gas-containing nanostructures can be modulated (e.g., increased, decreased, tuned, etc.) by altering the gas component of the fluid. In preferred aspects, oxygen is used in preparing the inventive electrokinetic fluids. In additional aspects mixtures of oxygen along with at least one other gas selected from Nitrogen, Oxygen, Argon, Carbon dioxide, Neon, Helium, krypton, hydrogen and Xenon. As described above, the ions may also be varied, including along with varying the gas constitutent(s).

Given the teachings and assay systems disclosed herein (e.g., cell-based cytokine assays, patch-clamp assays, etc.) one of skill in the art will readily be able to select appropriate salts and concentrations thereof to achieve the biological activities disclosed herein.

TABLE 1
Exemplary cations and anions.
Common Cations:

| Name | Formula | Other name(s) |
|---|---|---|
| Aluminum | Al⁺³ | |
| Ammonium | NH₄⁺ | |
| Barium | Ba⁺² | |
| Calcium | Ca⁺² | |
| Chromium(II) | Cr⁺² | Chromous |
| Chromium(III) | Cr⁺³ | Chromic |
| Copper(I) | Cu⁺ | Cuprous |
| Copper(II) | Cu⁺² | Cupric |
| Iron(II) | Fe⁺² | Ferrous |
| Iron(III) | Fe⁺³ | Ferric |
| Hydrogen | H⁺ | |
| Hydronium | H₃O⁺ | |
| Lead(II) | Pb⁺² | |
| Lithium | Li⁺ | |
| Magnesium | Mg⁺² | |
| Manganese(II) | Mn⁺² | Manganous |
| Manganese(III) | Mn⁺³ | Manganic |
| Mercury(I) | Hg₂⁺² | Mercurous |
| Mercury(II) | Hg⁺² | Mercuric |
| Nitronium | NO₂⁺ | |
| Potassium | K⁺ | |
| Silver | Ag⁺ | |
| Sodium | Na⁺ | |
| Strontium | Sr⁺² | |
| Tin(II) | Sn⁺² | Stannous |
| Tin(IV) | Sn⁺⁴ | Stannic |
| Zinc | Zn⁺² | |

Common Anions:
Simple ions:

| | |
|---|---|
| Hydride | H⁻ |
| Fluoride | F⁻ |
| Chloride | Cl⁻ |
| Bromide | Br⁻ |
| Iodide | I⁻ |
| Oxide | O²⁻ |
| Sulfide | S²⁻ |
| Nitride | N³⁻ |

Oxoanions:

| | |
|---|---|
| Arsenate | AsO₄³⁻ |
| Arsenite | AsO₃³⁻ |
| Sulfate | SO₄²⁻ |
| Hydrogen sulfate | HSO₄⁻ |
| Thiosulfate | S₂O₃²⁻ |
| Sulfite | SO₃²⁻ |
| Perchlorate | ClO₄⁻ |
| Chlorate | ClO₃⁻ |
| Chlorite | ClO₂⁻ |
| Hypochlorite | OCl⁻ |
| Carbonate | CO₃²⁻ |
| Hydrogen carbonate | HCO₃⁻ |
| or Bicarbonate | |
| Phosphate | PO₄³⁻ |
| Hydrogen phosphate | HPO₄²⁻ |
| Dihydrogen phosphate | H₂PO₄⁻ |
| Nitrate | NO₃⁻ |
| Nitrite | NO₂⁻ |
| Iodate | IO₃⁻ |
| Bromate | BrO₃⁻ |
| Hypobromite | OBr⁻ |
| Chromate | CrO₄²⁻ |
| Dichromate | Cr₂O₇²⁻ |

Anions from Organic Acids:

| | |
|---|---|
| Acetate | CH₃COO⁻ |
| formate | HCOO⁻ |

Others:

| | |
|---|---|
| Cyanide | CN⁻ |
| Cyanate | OCN⁻ |
| Thiocyanate | SCN⁻ |
| Hydroxide | OH⁻ |
| Amide | NH₂⁻ |
| Peroxide | O₂²⁻ |
| Oxalate | C₂O₄²⁻ |
| Permanganate | MnO₄⁻ |

TABLE 2
Exemplary cations and anions.

| | | |
|---|---|---|
| Formula | Charge | Name |

Monoatomic Cations

| | | |
|---|---|---|
| H⁺ | 1+ | hydrogen ion |
| Li⁺ | 1+ | lithium ion |
| Na⁺ | 1+ | sodium ion |
| K⁺ | 1+ | potassium ion |
| Cs⁺ | 1+ | cesium ion |
| Ag⁺ | 1+ | silver ion |
| Mg²⁺ | 2+ | magnesium ion |
| Ca²⁺ | 2+ | calcium ion |
| Sr²⁺ | 2+ | strontium ion |
| Ba²⁺ | 2+ | barium ion |
| Zn²⁺ | 2+ | zinc ion |
| Cd²⁺ | 2+ | cadmium ion |
| Al³⁺ | 3+ | aluminum ion |

Polyatomic Cations

| | | |
|---|---|---|
| NH₄⁺ | 1+ | ammonium ion |
| H₃O⁺ | 1+ | hydronium ion |

Multivalent Cations

| | | |
|---|---|---|
| Cr²⁺ | 2 | chromium(II) or chromous ion |
| Cr³⁺ | 3 | chromium(III)or chromic ion |
| Mn²⁺ | 2 | manganese(II) or manganous ion |
| Mn⁴⁺ | 4 | manganese(IV) ion |
| Fe²⁺ | 2 | iron(II) or ferrous ion |
| Fe³⁺ | 3 | iron(III) or ferric ion |
| Co²⁺ | 2 | cobalt(II) or cobaltous ion |
| Co³⁺ | 3 | cobalt(II) or cobaltic ion |
| Ni²⁺ | 2 | nickel(II) or nickelous ion |
| Ni³⁺ | 3 | nickel(III) or nickelic ion |
| Cu⁺ | 1 | copper(I) or cuprous ion |
| Cu²⁺ | 2 | copper(II) or cupric ion |
| Sn²⁺ | 2 | tin(II) or atannous ion |
| Sn⁴⁺ | 4 | tin(IV) or atannic ion |
| Pb²⁺ | 2 | lead(II) or plumbous ion |
| Pb⁴⁺ | 4 | lead(IV) or plumbic ion |

Monoatomic Anions

| | | |
|---|---|---|
| H⁻ | 1− | hydride ion |
| F⁻ | 1− | fluoride ion |
| Cl⁻ | 1− | chloride ion |
| Br⁻ | 1− | bromide ion |
| I⁻ | 1− | iodide ion |
| O²⁻ | 2− | oxide ion |
| S²⁻ | 2− | sulfide ion |
| N³⁻ | 3− | nitride ion |

Polyatomic Anions

| | | |
|---|---|---|
| OH⁻ | 1− | hydroxide ion |
| CN⁻ | 1− | cyanide ion |
| SCN⁻ | 1− | thiocyanate ion |
| C₂H₃O₂⁻ | 1− | acetate ion |
| ClO⁻ | 1− | hypochlorite ion |
| ClO₂⁻ | 1− | chlorite ion |
| ClO₃⁻ | 1− | chlorate ion |
| ClO₄⁻ | 1− | perchlorate ion |
| NO₂⁻ | 1− | nitrite ion |
| NO₃⁻ | 1− | nitrate ion |
| MnO₄²⁻ | 2− | permanganate ion |
| CO₃²⁻ | 2− | carbonate ion |
| C₂O₄²⁻ | 2− | oxalate ion |
| CrO₄²⁻ | 2− | chromate ion |
| Cr₂O₇²⁻ | 2− | dichromate ion |
| SO₃²⁻ | 2− | sulfite ion |
| SO₄²⁻ | 2− | sulfate ion |
| PO₃³⁻ | 3− | phosphite ion |
| PO₄³⁻ | 3− | phosphate ion |

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media) useful in the treatment of diabetes or diabetes related disorders. A medium, or media, is termed “minimal” if it only contains the nutrients essential for growth. For prokaryotic host cells, a minimal media typically includes a source of carbon, nitrogen, phosphorus, magnesium, and trace amounts of iron and calcium. (Gunsalus and Stanter, The Bacteria, V. 1, Ch. 1 Acad. Press Inc., N.Y. (1960)). Most minimal media use glucose as a carbon source, ammonia as a nitrogen source, and orthophosphate (e.g., PO₄) as the phosphorus source. The media components can be varied or supplemented according to the specific prokaryotic or eukaryotic organism(s) grown, in order to encourage optimal growth without inhibiting target protein production. (Thompson et al., Biotech. and Bioeng. 27: 818-824 (1985)).

In particular aspects, the electrokinetically altered aqueous fluids are suitable to modulate ¹³C-NMR line-widths of reporter solutes (e.g., Trehelose) dissolved therein. NMR line-width effects are in indirect method of measuring, for example, solute ‘tumbling’ in a test fluid as described herein in particular working Examples.

In particular aspects, the electrokinetically altered aqueous fluids are characterized by at least one of: distinctive square wave voltametry peak differences at any one of −0.14V, −0.47V, −1.02V and −1.36V; polarographic peaks at −0.9 volts; and an absence of polarographic peaks at −0.19 and −0.3 volts, which are unique to the electrokinetically generated fluids as disclosed herein in particular working Examples.

In particular aspects, the electrokinetically altered aqueous fluids are suitable to alter cellular membrane conductivity (e.g., a voltage-dependent contribution of the whole-cell conductance as measure in patch clamp studies disclosed herein).

In particular aspects, the electrokinetically altered aqueous fluids are oxygenated, wherein the oxygen in the fluid is present in an amount of at least 15, ppm, at least 25 ppm, at least 30 ppm, at least 40 ppm, at least 50 ppm, or at least 60 ppm dissolved oxygen at atmospheric pressure. In particular aspects, the electrokinetically altered aqueous fluids have less than 15 ppm, less than 10 ppm of dissolved oxygen at atmospheric pressure, or approximately ambient oxygen levels.

In particular aspects, the electrokinetically altered aqueous fluids are oxygenated, wherein the oxygen in the fluid is present in an amount between approximately 8 ppm and approximately 15 ppm, and in this case is sometimes referred to herein as “Solas.” In particular aspects, the electrokinetically altered aqueous fluid comprises at least one of solvated electrons (e.g., stabilized by molecular oxygen), and electrokinetically modified and/or charged oxygen species, and wherein in certain embodiments the solvated electrons and/or electrokinetically modified or charged oxygen species are present in an amount of at least 0.01 ppm, at least 0.1 ppm, at least 0.5 ppm, at least 1 ppm, at least 3 ppm, at least 5 ppm, at least 7 ppm, at least 10 ppm, at least 15 ppm, or at least 20 ppm.

In particular aspects, the electrokinetically altered aqueous fluids are characterized by differential (e.g., increased or decreased) permittivity relative to control, non-electrokinetically altered fluids. In preferred aspects, the electrokinetically altered aqueous fluids are characterized by differential, increased permittivity relative to control, non-electrokinetically altered fluids. Permittivity (∈) (farads per meter) is a measure of the ability of a material to be polarized by an electric field and thereby reduce the total electric field inside the material. Thus, permittivity relates to a material's ability to transmit (or “permit”) an electric field. Capacitance (C) (farad; coulomb per volt), a closely related property, is a measure of the ability of a material to hold charge if a voltage is applied across it (e.g., best modeled by a dielectric layer sandwiched between two parallel conductive plates). If a voltage V is applied across a capacitor of capacitance C, then the charge Q that it can hold is directly proportional to the applied voltage V, with the capacitance C as the proportionality constant. Thus, Q=CV, or C=Q/V. The capacitance of a capacitor depends on the permittivity ∈ of the dielectric layer, as well as the area A of the capacitor and the separation distance d between the two conductive plates. Permittivity and capacitance are mathematically related as follows: C=∈ (A/d). When the dielectric used is vacuum, then the capacitance Co=∈o (A/d), where co is the permittivity of vacuum (8.85×10⁻¹² F/m). The dielectric constant (k), or relative permittivity of a material is the ratio of its permittivity ∈ to the permittivity of vacuum ∈o, so k=∈/∈o (the dielectric constant of vacuum is 1). A low-k dielectric is a dielectric that has a low permittivity, or low ability to polarize and hold charge. A high-k dielectric, on the other hand, has a high permittivity. Because high-k dielectrics are good at holding charge, they are the preferred dielectric for capacitors. High-k dielectrics are also used in memory cells that store digital data in the form of charge.

In particular aspects, the electrokinetically altered aqueous fluids are suitable to alter cellular membrane structure or function (e.g., altering of a conformation, ligand binding activity, or a catalytic activity of a membrane associated protein) sufficient to provide for modulation of intracellular signal transduction, wherein in particular aspects, the membrane associated protein comprises at least one selected from the group consisting of receptors, transmembrane receptors (e.g., G-Protein Coupled Receptor (GPCR), TSLP receptor, beta 2 adrenergic receptor, bradykinin receptor, etc.), ion channel proteins, intracellular attachment proteins, cellular adhesion proteins, and integrins. In certain aspects, the effected G-Protein Coupled Receptor (GPCR) interacts with a G protein a subunit (e.g., Gαₛ, Gαᵢ, Gα_{q}, and Gα₁₂).

In particular aspects, the electrokinetically altered aqueous fluids are suitable to modulate intracellular signal transduction, comprising modulation of a calcium dependent cellular messaging pathway or system (e.g., modulation of phospholipase C activity, or modulation of adenylate cyclase (AC) activity).

In particular aspects, the electrokinetically altered aqueous fluids are characterized by various biological activities (e.g., regulation of cytokines, receptors, enzymes and other proteins and intracellular signaling pathways) described in the working Examples and elsewhere herein.

In particular aspects, the electrokinetically altered aqueous fluids display synergy with glatiramer acetate interferon-β, mitoxantrone, and/or natalizumab. In particular aspects, the electrokinetically altered aqueous fluids reduce DEP-induced TSLP receptor expression in bronchial epithelial cells (BEC).

In particular aspects, the electrokinetically altered aqueous fluids inhibit the DEP-induced cell surface-bound MMP9 levels in bronchial epithelial cells (BEC).

In particular aspects, the biological effects of the electrokinetically altered aqueous fluids are inhibited by diphtheria toxin, indicating that beta blockade, GPCR blockade and Ca channel blockade affects the activity of the electrokinetically altered aqueous fluids (e.g., on regulatory T cell function).

In particular aspects, the physical and biological effects (e.g., the ability to alter cellular membrane structure or function sufficient to provide for modulation of intracellular signal transduction) of the electrokinetically altered aqueous fluids persists for at least two, at least three, at least four, at least five, at least 6 months, or longer periods, in a closed container (e.g., closed gas-tight container).

Therefore, further aspects provide said electrokinetically-generated solutions and methods of producing an electrokinetically altered oxygenated aqueous fluid or solution, comprising: providing a flow of a fluid material between two spaced surfaces in relative motion and defining a mixing volume therebetween, wherein the dwell time of a single pass of the flowing fluid material within and through the mixing volume is greater than 0.06 seconds or greater than 0.1 seconds; and introducing oxygen (O₂) into the flowing fluid material within the mixing volume under conditions suitable to dissolve at least 20 ppm, at least 25 ppm, at least 30, at least 40, at least 50, or at least 60 ppm oxygen into the material, and electrokinetically alter the fluid or solution. In certain aspects, the oxygen is infused into the material in less than 100 milliseconds, less than 200 milliseconds, less than 300 milliseconds, or less than 400 milliseconds. In particular embodiments, the ratio of surface area to the volume is at least 12, at least 20, at least 30, at least 40, or at least 50.

Yet further aspects, provide a method of producing an electrokinetically altered oxygenated aqueous fluid or solution, comprising: providing a flow of a fluid material between two spaced surfaces defining a mixing volume therebetween; and introducing oxygen into the flowing material within the mixing volume under conditions suitable to infuse at least 20 ppm, at least 25 ppm, at least 30, at least 40, at least 50, or at least 60 ppm oxygen into the material in less than 100 milliseconds, less than 200 milliseconds, less than 300 milliseconds, or less than 400 milliseconds. In certain aspects, the dwell time of the flowing material within the mixing volume is greater than 0.06 seconds or greater than 0.1 seconds. In particular embodiments, the ratio of surface area to the volume is at least 12, at least 20, at least 30, at least 40, or at least 50.

Additional embodiments provide a method of producing an electrokinetically altered oxygenated aqueous fluid or solution, comprising use of a mixing device for creating an output mixture by mixing a first material and a second material, the device comprising: a first chamber configured to receive the first material from a source of the first material; a stator; a rotor having an axis of rotation, the rotor being disposed inside the stator and configured to rotate about the axis of rotation therein, at least one of the rotor and stator having a plurality of through-holes; a mixing chamber defined between the rotor and the stator, the mixing chamber being in fluid communication with the first chamber and configured to receive the first material therefrom, and the second material being provided to the mixing chamber via the plurality of through-holes formed in the one of the rotor and stator; a second chamber in fluid communication with the mixing chamber and configured to receive the output material therefrom; and a first internal pump housed inside the first chamber, the first internal pump being configured to pump the first material from the first chamber into the mixing chamber. In certain aspects, the first internal pump is configured to impart a circumferential velocity into the first material before it enters the mixing chamber.

Further embodiments provide a method of producing an electrokinetically altered oxygenated aqueous fluid or solution, comprising use of a mixing device for creating an output mixture by mixing a first material and a second material, the device comprising: a stator; a rotor having an axis of rotation, the rotor being disposed inside the stator and configured to rotate about the axis of rotation therein; a mixing chamber defined between the rotor and the stator, the mixing chamber having an open first end through which the first material enters the mixing chamber and an open second end through which the output material exits the mixing chamber, the second material entering the mixing chamber through at least one of the rotor and the stator; a first chamber in communication with at least a majority portion of the open first end of the mixing chamber; and a second chamber in communication with the open second end of the mixing chamber.

Additional aspects provide an electrokinetically altered oxygenated aqueous fluid or solution made according to any of the above methods. In particular aspects the administered inventive electrokinetically altered fluids comprise charge-stabilized oxygen-containing nanostructures in an amount sufficient to provide modulation of at least one of cellular membrane potential and cellular membrane conductivity. In certain embodiments, the electrokinetically altered fluids are superoxygenated (e.g., RNS-20, RNS-40 and RNS-60, comprising 20 ppm, 40 ppm and 60 ppm dissolved oxygen, respectively, in standard saline). In particular embodiments, the electrokinetically altered fluids are not-superoxygenated (e.g., RNS-10 or Solas, comprising 10 ppm (e.g., approx. ambient levels of dissolved oxygen in standard saline). In certain aspects, the salinity, sterility, pH, etc., of the inventive electrokinetically altered fluids is established at the time of electrokinetic production of the fluid, and the sterile fluids are administered by an appropriate route. Alternatively, at least one of the salinity, sterility, pH, etc., of the fluids is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible therewith.

The present disclosure sets forth novel gas-enriched fluids, including, but not limited to gas-enriched ionic aqueous solutions, aqueous saline solutions (e.g., standard aqueous saline solutions, and other saline solutions as discussed herein and as would be recognized in the art, including any physiological compatible saline solutions), cell culture media (e.g., minimal medium, and other culture media).

According to particular aspects of the methods and fluids above, the charge-stabilized oxygen-containing nanostructures comprise charge-stabilized oxygen-containing nanobubbles predominantly having an average diameter less than 100 nm. According to particular aspects, the charge-stabilized oxygen-containing nanobubbles are stable to persist in solution for at least months in a closed container at atmospheric pressure.

### Methods of Treatment

The term “treating” or “administering” refers to, and includes, reversing, alleviating, inhibiting the progress of, or preventing a disease, disorder or condition, or one or more symptoms thereof and “treatment” and “therapeutically” refer to the act of treating, as defined herein.

A “therapeutically effective amount” is any amount of any of the compounds utilized in the course of practicing the invention provided herein that is sufficient to reverse, alleviate, inhibit the progress of, or prevent a disease, disorder or condition, or one or more symptoms thereof.

Certain embodiments herein relate to therapeutic compositions and methods of treatment for a subject by enhancing neuronal transmission, as disclosed herein.

### Combination Therapy:

Additional aspects provide the herein disclosed inventive methods, further comprising combination therapy, wherein at least one additional therapeutic agent is administered to the patient. In certain aspects, the at least one additional therapeutic agent is an anti-inflammatory agent.

### Exemplary Relevant Molecular Interactions:

Conventionally, quantum properties are thought to belong to elementary particles of less than 10⁻¹⁰ meters, while the macroscopic world of our everyday life is referred to as classical, in that it behaves according to Newton's laws of motion.

Recently, molecules have been described as forming clusters that increase in size with dilution. These clusters measure several micrometers in diameter, and have been reported to increase in size non-linearly with dilution. Quantum coherent domains measuring 100 nanometers in diameter have been postulated to arise in pure water, and collective vibrations of water molecules in the coherent domain may eventually become phase locked to electromagnetic field fluctuations, providing for stable oscillations in water, providing a form of ‘memory’ in the form of excitation of long lasting coherent oscillations specific to dissolved substances in the water that change the collective structure of the water, which may in turn determine the specific coherent oscillations that develop. Where these oscillations become stabilized by magnetic field phase coupling, the water, upon dilution may still carry ‘seed’ coherent oscillations. As a cluster of molecules increases in size, its electromagnetic signature is correspondingly amplified, reinforcing the coherent oscillations carried by the water.

Despite variations in the cluster size of dissolved molecules and detailed microscopic structure of the water, a specificity of coherent oscillations may nonetheless exist. One model for considering changes in properties of water is based on considerations involved in crystallization.

A protonated water cluster typically takes the form of H⁺ (H₂0)ₙ. Some protonated water clusters occur naturally, such as in the ionosphere. Without being bound by any particular theory, and according to particular aspects, other types of water clusters or structures (nanoclusters, nanocages, nanobubbles) are possible, including nanostructures comprising oxygen (and possibly stabilized electrons imparted to the inventive output materials). Oxygen atoms may be caught in the resulting structures. The chemistry of the semi-bound nanocage or nanobubble allows the oxygen and/or stabilized electrons to remain dissolved for extended periods of time. Other atoms or molecules, such as medicinal compounds, can be combined for sustained delivery purposes. The specific chemistry of the solution material and dissolved compounds depend on the interactions of those materials.

As described previously in Applicants' WO 2009/055729, “Double Layer Effect,” “Dwell Time,” “Rate of Infusion,” and “Bubble size Measurements,” the electrokinetic mixing device creates, in a matter of milliseconds, a unique non-linear fluid dynamic interaction of the first material and the second material with complex, dynamic turbulence providing complex mixing in contact with an effectively enormous surface area (including those of the device and of the exceptionally small gas bubbles; nanobubbles of less than 100 nm) that provides for the novel therapeutic effects described herein. Additionally, feature-localized electrokinetic effects (voltage/current) were demonstrated using a specially designed mixing device comprising insulated rotor and stator features (also see, e.g., Applicants' issued U.S. Pat. Nos. 7,832,920; 7,919,534; 8,410,182; 8,445,546; 8,449,172; and 8,470,893, all incorporated herein by reference in their respective entireties).

As recognized in the art, charge redistributions and/or solvated electrons are known to be highly unstable in aqueous solution. According to particular aspects, Applicants' electrokinetic effects (e.g., charge redistributions, including, in particular aspects, solvated electrons) are surprisingly stabilized within the output material (e.g., saline solutions, ionic solutions). In fact, as described herein, the stability of the properties and biological activity of the inventive electrokinetic fluids (e.g., RNS-60 or Solas (processed through device but with no added Oxygen) can be maintained for months in a gas-tight container, indicating involvement of dissolved gas (e.g., oxygen) in helping to generate and/or maintain, and/or mediate the properties and activities of the inventive solutions. Significantly, the charge redistributions and/or solvated electrons are stably configured in the inventive electrokinetic ionic aqueous fluids in an amount sufficient to provide, upon contact with a living cell (e.g., mammalian cell) by the fluid, modulation of at least one of cellular membrane potential and cellular membrane conductivity (see, e.g., cellular patch clamp working Example 23 from WO 2009/055729 and as disclosed herein).

As described herein under “Molecular Interactions,” to account for the stability and biological compatibility of the inventive electrokinetic fluids (e.g., electrokinetic saline solutions), Applicants have proposed that interactions between the water molecules and the molecules of the substances (e.g., oxygen) dissolved in the water change the collective structure of the water and provide for nanoscale structures (e.g., nanobubbles), including nanostructure (e.g., nanobubbles) comprising oxygen and/or stabilized electrons imparted to the inventive output materials. Without being bound by mechanism, the configuration of the nanostructures (e.g., nanobubbles) in particular aspects is such that they: comprise (at least for formation and/or stability and/or biological activity) dissolved gas (e.g., oxygen); enable the electrokinetic fluids (e.g., RNS-60 or Solas saline fluids) to modulate (e.g., impart or receive) charges and/or charge effects upon contact with a cell membrane or related constituent thereof; and in particular aspects provide for stabilization (e.g., carrying, harboring, trapping) solvated electrons in a biologically-relevant form.

According to particular aspects, and as supported by the present disclosure, in ionic or saline (e.g., standard saline, NaCl) solutions, the inventive nanostructures comprise charge-stabilized nanostructures (e.g., nanobubbles) (e.g., average diameter less than 100 nm) that may comprise at least one dissolved gas molecule (e.g., oxygen) within a charge-stabilized hydration shell. According to additional aspects, the charge-stabilized hydration shell may comprise a cage or void harboring the at least one dissolved gas molecule (e.g., oxygen). According to further aspects, by virtue of the provision of suitable charge-stabilized hydration shells, the charge-stabilized nanostructure and/or charge-stabilized oxygen containing nanostructures may additionally comprise a solvated electron (e.g., stabilized solvated electron).

According to particular aspects of the present invention, Applicants' novel electrokinetic fluids comprise a novel, biologically active form of charge-stabilized oxygen-containing nanostructures (e.g., nanobubbles), and may further comprise novel arrays, clusters or associations of such structures (e.g., of such nanobubbles).

According to a charge-stabilized microbubble model, the short-range molecular order of the water structure is destroyed by the presence of a gas molecule (e.g., a dissolved gas molecule initially complexed with a nonadsorptive ion provides a short-range order defect), providing for condensation of ionic droplets, wherein the defect is surrounded by first and second coordination spheres of water molecules, which are alternately filled by adsorptive ions (e.g., acquisition of a ‘screening shell of Na⁺ ions to form an electrical double layer) and nonadsorptive ions (e.g., Cl⁻ ions occupying the second coordination sphere) occupying six and 12 vacancies, respectively, in the coordination spheres. In under-saturated ionic solutions (e.g., undersaturated saline solutions), this hydrated ‘nucleus’ remains stable until the first and second spheres are filled by six adsorptive and five nonadsorptive ions, respectively, and then undergoes Coulomb explosion creating an internal void containing the gas molecule, wherein the adsorptive ions (e.g., Na⁺ ions) are adsorbed to the surface of the resulting void, while the nonadsorptive ions (or some portion thereof) diffuse into the solution (Bunkin et al., supra). In this model, the void in the nanostructure is prevented from collapsing by Coulombic repulsion between the ions (e.g., Na⁺ ions) adsorbed to its surface. The stability of the void-containing nanostructures is postulated to be due to the selective adsorption of dissolved ions with like charges onto the void/bubble surface and diffusive equilibrium between the dissolved gas and the gas inside the bubble, where the negative (outward electrostatic pressure exerted by the resulting electrical double layer provides stable compensation for surface tension, and the gas pressure inside the bubble is balanced by the ambient pressure. According to the model, formation of such microbubbles requires an ionic component, and in certain aspects collision-mediated associations between particles may provide for formation of larger order clusters (arrays) (Id).

The charge-stabilized microbubble model suggests that the particles can be gas microbubbles, but contemplates only spontaneous formation of such structures in ionic solution in equilibrium with ambient air, is uncharacterized and silent as to whether oxygen is capable of forming such structures, and is likewise silent as to whether solvated electrons might be associated and/or stabilized by such structures.

According to particular aspects, the inventive electrokinetic fluids comprising charge-stabilized nanostructures and/or charge-stabilized oxygen-containing nanostructures are novel and fundamentally distinct from the postulated non-electrokinetic, atmospheric charge-stabilized microbubble structures according to the microbubble model. Significantly, this conclusion is unavoidable, deriving, at least in part, from the fact that control saline solutions do not have the biological properties disclosed herein, whereas Applicants' charge-stabilized nanostructures provide a novel, biologically active form of charge-stabilized oxygen-containing nanostructures.

According to particular aspects of the present invention, Applicants' novel electrokinetic device and methods provide for novel electrokinetically altered fluids comprising significant quantities of charge-stabilized nanostructures in excess of any amount that may or may not spontaneously occur in ionic fluids in equilibrium with air, or in any non-electrokinetically generated fluids. In particular aspects, the charge-stabilized nanostructures comprise charge-stabilized oxygen-containing nanostructures. In additional aspects, the charge-stabilized nanostructures are all, or substantially all charge-stabilized oxygen-containing nanostructures, or the charge-stabilized oxygen-containing nanostructures the major charge-stabilized gas-containing nanostructure species in the electrokinetic fluid.

According to yet further aspects, the charge-stabilized nanostructures and/or the charge-stabilized oxygen-containing nanostructures may comprise or harbor a solvated electron, and thereby provide a novel stabilized solvated electron carrier. In particular aspects, the charge-stabilized nanostructures and/or the charge-stabilized oxygen-containing nanostructures provide a novel type of electride (or inverted electride), which in contrast to conventional solute electrides having a single organically coordinated cation, rather have a plurality of cations stably arrayed about a void or a void containing an oxygen atom, wherein the arrayed sodium ions are coordinated by water hydration shells, rather than by organic molecules. According to particular aspects, a solvated electron may be accommodated by the hydration shell of water molecules, or preferably accommodated within the nanostructure void distributed over all the cations. In certain aspects, the inventive nanostructures provide a novel ‘super electride’ structure in solution by not only providing for distribution/stabilization of the solvated electron over multiple arrayed sodium cations, but also providing for association or partial association of the solvated electron with the caged oxygen molecule(s) in the void—the solvated electron distributing over an array of sodium atoms and at least one oxygen atom. According to particular aspects, therefore, ‘solvated electrons’ as presently disclosed in association with the inventive electrokinetic fluids, may not be solvated in the traditional model comprising direct hydration by water molecules. Alternatively, in limited analogy with dried electride salts, solvated electrons in the inventive electrokinetic fluids may be distributed over multiple charge-stabilized nanostructures to provide a ‘lattice glue’ to stabilize higher order arrays in aqueous solution.

In particular aspects, the inventive charge-stabilized nanostructures and/or the charge-stabilized oxygen-containing nanostructures are capable of interacting with cellular membranes or constituents thereof, or proteins, etc., to mediate biological activities. In particular aspects, the inventive charge-stabilized nanostructures and/or the charge-stabilized oxygen-containing nanostructures harboring a solvated electron are capable of interacting with cellular membranes or constituents thereof, or proteins, etc., to mediate biological activities.

In particular aspects, the inventive charge-stabilized nanostructures and/or the charge-stabilized oxygen-containing nanostructures interact with cellular membranes or constituents thereof, or proteins, etc., as a charge and/or charge affect donor (delivery) and/or as a charge and/or charge effect recipient to mediate biological activities. In particular aspects, the inventive charge-stabilized nanostructures and/or the charge-stabilized oxygen-containing nanostructures harboring a solvated electron interact with cellular membranes as a charge and/or charge effect donor and/or as a charge and/or charge effect recipient to mediate biological activities.

In particular aspects, the inventive charge-stabilized nanostructures and/or the charge-stabilized oxygen-containing nanostructures are consistent with, and account for the observed stability and biological properties of the inventive electrokinetic fluids.

In particular aspects, the charge-stabilized oxygen-containing nanostructures substantially comprise, take the form of, or can give rise to, charge-stabilized oxygen-containing nanobubbles. In particular aspects, charge-stabilized oxygen-containing clusters provide for formation of relatively larger arrays of charge-stabilized oxygen-containing nanostructures, and/or charge-stabilized oxygen-containing nanobubbles or arrays thereof. In particular aspects, the charge-stabilized oxygen-containing nanostructures can provide for formation of hydrophobic nanobubbles upon contact with a hydrophobic surface.

In particular aspects, the charge-stabilized oxygen-containing nanostructures substantially comprise at least one oxygen molecule. In certain aspects, the charge-stabilized oxygen-containing nanostructures substantially comprise at least 1, at least 2, at least 3, at least 4, at least 5, at least 10 at least 15, at least 20, at least 50, at least 100, or greater oxygen molecules. In particular aspects, charge-stabilized oxygen-containing nanostructures comprise or give rise to nanobubles (e.g., hydrophobid nanobubbles) of about 20 nm×1.5 nm, comprise about 12 oxygen molecules (e.g., based on the size of an oxygen molecule (approx 0.3 nm by 0.4 nm), assumption of an ideal gas and application of n=PV/RT, where P=1 atm, R=0.082 057 l.atm/mol.K; T=295K; V=pr²h=4.7×10⁻²² L, where r=10×10⁻⁹ m, h=1.5×10⁻⁹ m, and n=1.95×10⁻²² moles).

In certain aspects, the percentage of oxygen molecules present in the fluid that are in such nanostructures, or arrays thereof, having a charge-stabilized configuration in the ionic aqueous fluid is a percentage amount selected from the group consisting of greater than: 0.1%, 1%; 2%; 5%; 10%; 15%; 20%; 25%; 30%; 35%; 40%; 45%; 50%; 55%; 60%; 65%; 70%; 75%; 80%; 85%; 90%; and greater than 95%. Preferably, this percentage is greater than about 5%, greater than about 10%, greater than about 15% f, or greater than about 20%. In additional aspects, the substantial size of the charge-stabilized oxygen-containing nanostructures, or arrays thereof, having a charge-stabilized configuration in the ionic aqueous fluid is a size selected from the group consisting of less than: 100 nm; 90 nm; 80 nm; 70 nm; 60 nm; 50 nm; 40 nm; 30 nm; 20 nm; 10 nm; 5 nm; 4 nm; 3 nm; 2 nm; and 1 nm. Preferably, this size is less than about 50 nm, less than about 40 nm, less than about 30 nm, less than about 20 nm, or less than about 10 nm.

In certain aspects, the inventive electrokinetic fluids comprise solvated electrons. In further aspects, the inventive electrokinetic fluids comprises charge-stabilized nanostructures and/or charge-stabilized oxygen-containing nanostructures, and/or arrays thereof, which comprise at least one of: solvated electron(s); and unique charge distributions (polar, symmetric, asymmetric charge distribution). In certain aspects, the charge-stabilized nanostructures and/or charge-stabilized oxygen-containing nanostructures, and/or arrays thereof, have paramagnetic properties.

By contrast, relative to the inventive electrokinetic fluids, control pressure pot oxygenated fluids (non-electrokinetic fluids) and the like do not comprise such electrokinetically generated charge-stabilized biologically-active nanostructures and/or biologically-active charge-stabilized oxygen-containing nanostructures and/or arrays thereof, capable of modulation of at least one of cellular membrane potential and cellular membrane conductivity.

### Routes and Forms of Administration

In particular exemplary embodiments, the gas-enriched fluid of the present invention may function as a therapeutic composition alone or in combination with another therapeutic agent such that the therapeutic composition enhances neuronal transmission. The therapeutic compositions of the present invention include compositions that are able to be administered to a subject in need thereof. In certain embodiments, the therapeutic composition formulation may also comprise at least one additional agent selected from the group consisting of: carriers, adjuvants, emulsifying agents, suspending agents, sweeteners, flavorings, perfumes, and binding agents.

As used herein, “pharmaceutically acceptable carrier” and “carrier” generally refer to a non-toxic, inert solid, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In particular aspects, such carriers and excipients may be gas-enriched fluids or solutions of the present invention.

The pharmaceutically acceptable carriers described herein, for example, vehicles, adjuvants, excipients, or diluents, are well known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

In addition to the therapeutic gas-enriched fluid of the present invention, the therapeutic composition may further comprise inert diluents such as additional non-gas-enriched water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. As is appreciated by those of ordinary skill, a novel and improved formulation of a particular therapeutic composition, a novel gas-enriched therapeutic fluid, and a novel method of delivering the novel gas-enriched therapeutic fluid may be obtained by replacing one or more inert diluents with a gas-enriched fluid of identical, similar, or different composition. For example, conventional water may be replaced or supplemented by a gas-enriched fluid produced by mixing oxygen into water or deionized water to provide gas-enriched fluid.

In certain embodiments, the inventive gas-enriched fluid may be combined with one or more therapeutic agents and/or used alone. In particular embodiments, incorporating the gas-enriched fluid may include replacing one or more solutions known in the art, such as deionized water, saline solution, and the like with one or more gas-enriched fluid, thereby providing an improved therapeutic composition for delivery to the subject.

Certain embodiments provide for therapeutic compositions comprising a gas-enriched fluid of the present invention, a pharmaceutical composition or other therapeutic agent or a pharmaceutically acceptable salt or solvate thereof, and at least one pharmaceutical carrier or diluent. These pharmaceutical compositions may be used in the prophylaxis and treatment of the foregoing diseases or conditions and in therapies as mentioned above. Preferably, the carrier must be pharmaceutically acceptable and must be compatible with, i.e., not have a deleterious effect upon, the other ingredients in the composition. The carrier may be a solid or liquid and is preferably formulated as a unit dose formulation, for example, a tablet that may contain from 0.05 to 95% by weight of the active ingredient.

Possible administration routes include oral, sublingual, buccal, parenteral (for example, subcutaneous, intramuscular, intra-arterial, intraperitoneally, intracisternally, intravesically, intrathecally, or intravenous), rectal, topical including transdermal, intravaginal, intraoccular, intraotical, intranasal, inhalation, and injection or insertion of implantable devices or materials.

### Administration Routes

Most suitable means of administration for a particular subject will depend on the nature and severity of the disease or condition being treated or the nature of the therapy being used, as well as the nature of the therapeutic composition or additional therapeutic agent. In certain embodiments, oral or topical administration is preferred.

Formulations suitable for oral administration may be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Additional formulations suitable for oral administration may be provided to include fine particle dusts or mists which may be generated by means of various types of metered dose pressurized aerosols, atomizers, nebulizers, or insufflators. In particular, powders or other compounds of therapeutic agents may be dissolved or suspended in a gas-enriched fluid of the present invention.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerin or sucrose acacia.

Formulations suitable for parenteral administration typically comprise sterile aqueous solutions containing a predetermined concentration of the active gas-enriched fluid and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for urethral, rectal or vaginal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Alternatively, colonic washes with the gas-enriched fluids of the present invention may be formulated for colonic or rectal administration.

Formulations suitable for topical, intraocular, intraotic, or intranasal application include ointments, creams, pastes, lotions, pastes, gels (such as hydrogels), sprays, dispersible powders and granules, emulsions, sprays or aerosols using flowing propellants (such as liposomal sprays, nasal drops, nasal sprays, and the like) and oils. Suitable carriers for such formulations include petroleum jelly, lanolin, polyethyleneglycols, alcohols, and combinations thereof. Nasal or intranasal delivery may include metered doses of any of these formulations or others. Likewise, intraotic or intraocular may include drops, ointments, irritation fluids and the like.

Formulations of the invention may be prepared by any suitable method, typically by uniformly and intimately admixing the gas-enriched fluid optionally with an active compound with liquids or finely divided solid carriers or both, in the required proportions and then, if necessary, shaping the resulting mixture into the desired shape.

For example a tablet may be prepared by compressing an intimate mixture comprising a powder or granules of the active ingredient and one or more optional ingredients, such as a binder, lubricant, inert diluent, or surface active dispersing agent, or by molding an intimate mixture of powdered active ingredient and a gas-enriched fluid of the present invention.

Suitable formulations for administration by inhalation include fine particle dusts or mists which may be generated by means of various types of metered dose pressurized aerosols, atomizers, nebulizers, or insufflators. In particular, powders or other compounds of therapeutic agents may be dissolved or suspended in a gas-enriched fluid of the present invention.

For pulmonary administration via the mouth, the particle size of the powder or droplets is typically in the range 0.5-10 μM, preferably 1-5 μM, to ensure delivery into the bronchial tree. For nasal administration, a particle size in the range 10-500 μM is preferred to ensure retention in the nasal cavity.

Metered dose inhalers are pressurized aerosol dispensers, typically containing a suspension or solution formulation of a therapeutic agent in a liquefied propellant. In certain embodiments, as disclosed herein, the gas-enriched fluids of the present invention may be used in addition to or instead of the standard liquefied propellant. During use, these devices discharge the formulation through a valve adapted to deliver a metered volume, typically from 10 to 150 μL, to produce a fine particle spray containing the therapeutic agent and the gas-enriched fluid. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol surfactants, such as oleic acid or sorbitan trioleate, anti-oxidants and suitable flavoring agents. Nebulizers are commercially available devices that transform solutions or suspensions of the active ingredient into a therapeutic aerosol mist either by means of acceleration of a compressed gas (typically air or oxygen) through a narrow venturi orifice, or by means of ultrasonic agitation. Suitable formulations for use in nebulizers consist of another therapeutic agent in a gas-enriched fluid and comprising up to 40% w/w of the formulation, preferably less than 20% w/w. In addition, other carriers may be utilized, such as distilled water, sterile water, or a dilute aqueous alcohol solution, preferably made isotonic with body fluids by the addition of salts, such as sodium chloride. Optional additives include preservatives, especially if the formulation is not prepared sterile, and may include methyl hydroxy-benzoate, anti-oxidants, flavoring agents, volatile oils, buffering agents and surfactants.

Suitable formulations for administration by insufflation include finely comminuted powders that may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

In addition to the ingredients specifically mentioned above, the formulations of the present invention may include other agents known to those skilled in the art, having regard for the type of formulation in issue. For example, formulations suitable for oral administration may include flavoring agents and formulations suitable for intranasal administration may include perfumes.

The therapeutic compositions of the invention can be administered by any conventional method available for use in conjunction with pharmaceutical drugs, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg. According to certain aspects daily dosage of active ingredient may be 0.001 liters to 10 liters, with the preferred dose being from about 0.01 liters to 1 liter.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Ointments, pastes, foams, occlusions, creams and gels also can contain excipients, such as starch, tragacanth, cellulose derivatives, silicones, bentonites, silica acid, and talc, or mixtures thereof. Powders and sprays also can contain excipients such as lactose, talc, silica acid, aluminum hydroxide, and calcium silicates, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such as nitrogen, carbon dioxide, and other inert gases. In addition, microspheres or nanoparticles may be employed with the gas-enriched therapeutic compositions or fluids of the present invention in any of the routes required to administer the therapeutic compounds to a subject.

The injection-use formulations can be presented in unit-dose or multi-dose sealed containers, such as ampules and vials, and can be stored in a freeze-dried (lyophilized) condition requiring only the addition of the sterile liquid excipient, or gas-enriched fluid, immediately prior to use. Extemporaneous injection solutions and suspensions can be prepared from sterile powders, granules, and tablets. The requirements for effective pharmaceutical carriers for injectable compositions are well known to those of ordinary skill in the art. See, for example, Pharmaceutics and Pharmacy Practice, J. B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising a gas-enriched fluid of the invention and optionally, an additional therapeutic and a flavor, usually sucrose and acacia or tragacanth; pastilles comprising a gas-enriched fluid and optional additional therapeutic agent in an inert base, such as gelatin and glycerin, or sucrose and acacia; and mouth washes or oral rinses comprising a gas-enriched fluid and optional additional therapeutic agent in a suitable liquid carrier; as well as creams, emulsions, gels and the like.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to a subject, especially an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the condition being treated. A suitable dose is that which will result in a concentration of the therapeutic composition in a subject that is known to affect the desired response.

The size of the dose also will be determined by the route, timing and frequency of administration as well as the existence, nature, and extent of any adverse side effects that might accompany the administration of the therapeutic composition and the desired physiological effect.

It will be appreciated that the compounds of the combination may be administered: (1) simultaneously by combination of the compounds in a co-formulation or (2) by alternation, i.e., delivering the compounds serially, sequentially, in parallel or simultaneously in separate pharmaceutical formulations. In alternation therapy, the delay in administering the second, and optionally a third active ingredient, should not be such as to lose the benefit of a synergistic therapeutic effect of the combination of the active ingredients. According to certain embodiments by either method of administration (1) or (2), ideally the combination should be administered to achieve the most efficacious results. In certain embodiments by either method of administration (1) or (2), ideally the combination should be administered to achieve peak plasma concentrations of each of the active ingredients. A one pill once-per-day regimen by administration of a combination co-formulation may be feasible for some patients suffering from inflammatory neurodegenerative diseases. According to certain embodiments effective peak plasma concentrations of the active ingredients of the combination will be in the range of approximately 0.001 to 100 μM. Optimal peak plasma concentrations may be achieved by a formulation and dosing regimen prescribed for a particular patient. It will also be understood that the inventive fluids and glatiramer acetate, interferon-beta, mitoxantrone, and/or natalizumab or the physiologically functional derivatives of any thereof, whether presented simultaneously or sequentially, may be administered individually, in multiples, or in any combination thereof. In general, during alternation therapy (2), an effective dosage of each compound is administered serially, where in co-formulation therapy (1), effective dosages of two or more compounds are administered together.

The combinations of the invention may conveniently be presented as a pharmaceutical formulation in a unitary dosage form. A convenient unitary dosage formulation contains the active ingredients in any amount from 1 mg to 1 g each, for example but not limited to, 10 mg to 300 mg. The synergistic effects of the inventive fluid in combination with glatiramer acetate, interferon-beta, mitoxantrone, and/or natalizumab may be realized over a wide ratio, for example 1:50 to 50:1 (inventive fluid: glatiramer acetate, interferon-beta, mitoxantrone, and/or natalizumab). In one embodiment the ratio may range from about 1:10 to 10:1. In another embodiment, the weight/weight ratio of inventive fluid to glatiramer acetate, interferon-beta, mitoxantrone, and/or natalizumab in a co-formulated combination dosage form, such as a pill, tablet, caplet or capsule will be about 1, i.e., an approximately equal amount of inventive fluid and glatiramer acetate, interferon-beta, mitoxantrone, and/or natalizumab. In other exemplary co-formulations, there may be more or less inventive fluid and glatiramer acetate, interferon-beta, mitoxantrone, and/or natalizumab. In one embodiment, each compound will be employed in the combination in an amount at which it exhibits anti-inflammatory activity when used alone. Other ratios and amounts of the compounds of said combinations are contemplated within the scope of the invention.

A unitary dosage form may further comprise inventive fluid and glatiramer acetate, interferon-beta, mitoxantrone, and/or natalizumab, or physiologically functional derivatives of either thereof, and a pharmaceutically acceptable carrier.

It will be appreciated by those skilled in the art that the amount of active ingredients in the combinations of the invention required for use in treatment will vary according to a variety of factors, including the nature of the condition being treated and the age and condition of the patient, and will ultimately be at the discretion of the attending physician or health care practitioner. The factors to be considered include the route of administration and nature of the formulation, the animal's body weight, age and general condition and the nature and severity of the disease to be treated.

It is also possible to combine any two of the active ingredients in a unitary dosage form for simultaneous or sequential administration with a third active ingredient. The three-part combination may be administered simultaneously or sequentially. When administered sequentially, the combination may be administered in two or three administrations. According to certain embodiments the three-part combination of inventive fluid and glatiramer acetate, interferon-beta, mitoxantrone, and/or natalizumab may be administered in any order.

The following examples are meant to be illustrative only and not limiting in any way.

### EXAMPLES

### Example 1

### Materials and Methods

Tau Proteins.

Recombinant human tau, h-tau42 (isoform with four tubulin binding motifs and two extra exons in the N-terminal domain) was isolated as previously described (Perez M, et al., In vitro assembly of tau protein: mapping the regions involved in filament formation. Biochemistry 40(20):5983-5991, 2001).

Immunohistochemistry.

A variation of the array tomography method by Micheva & Smith (Micheva K D & Smith S J., Array tomography: a new tool for imaging the molecular architecture and ultrastructure of neural circuits. Neuron 55(1):25-36, 2007) was followed. The ganglia were fixed by immersion in 4% paraformaldehyde (EM grade EM Sciences) plus 7.0% sucrose in calcium-free sea water for 3 hours; rinsed with 7% sucrose and 50 mM glycine in 0.1M PBS; dehydrated with graded ethanol dilutions (50%, 70%, 90% and 3×100%), embedded in LR White resin (medium grade, SPI), and polymerized in gelatin capsules at 53° C. for 24h. Semithin sections (500 nm) were mounted on subbed slides and encircled on the slides with a PAP pen (EM Sciences, USA). The immunocytochemistry was done as follows: a) blocked in 50 mM glycine in tris buffer pH 7.6, 5 min; b) primary antibody incubation, anti-tau PHF (AT8, Thermoscientific, USA) diluted 1:50 in 1% BSA in tris (trisBSA), 4 h; c) rinses in trisBSA 2×5 min: d) secondary antibody incubation, goat anti-mouse Alexa Fluor 594 diluted 1:150 in tris-BSA, 30 min; e) tris and distilled water rinses, 4×5 min each; f) mounting of slides with coverslips and anti-fading mounting media; g) image under fluorescent microscopy (Zeiss Axioimager, Germany). Controls were performed with the same protocol omitting the primary antibody. Primary antibody: Anti-tau PHF (AT8) (Thermoscientific, USA), secondary antibody: Alexa Fluor 594 goat anti-mouse (Invitrogen).

Electrophysiology and Microinjections.

The squid (Loligo paelli) stellate ganglia isolation from the mantle and the electrophysiological techniques used have been described previously (Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985). Two glass micropipette electrodes impaled the largest (most distal) presynaptic terminal digit at the synaptic junction site while the postsynaptic axon was impaled by one microelectrode at the junctional site. One of the pre-electrodes was used for pressure microinjection of h-Tau 42 and also supported voltage clamp current feedback, while the second monitored membrane potential. The total volume injected fluctuated between 0.1 and 1 pl. (Llinas, supra). The exact location of injection and the diffusion and steady-state distribution of the protein/fluorescent dye mix (0.001% dextran fluorescein) were monitored using a fluorescence microscope attached to a Hamamatsu camera system (Middlesex, N.J.). In all experiments a good correlation was observed between the localization of the fluorescence and the electrophysiological findings.

Electron Microscopy.

Immediately following the electrophysiological study the ganglia were removed from the recording chamber, fixed by immersion in glutaraldehyde, postfixed in osmium tetroxide, stained in block with uranium acetate, dehydrated and embedded in resin (Embed 812, EM Sciences). Ultrathin sections were collected on Pioloform (Ted Pella, Redding, Calif.) and carbon-coated single sloth grids, and contrasted with uranyl acetate and lead citrate. Morphometry and quantitative analysis of the synaptic vesicles were performed with the in house program developed with LabVIEW (National Instruments, Ostin Tex.). Electron micrographs were taken at an initial magnification of ×16,000 and ×31,500 and photographically enlarged to a magnification of ×40,000 and ×79,000 for synaptic vesicles and clathrin-coated vesicle (CCV) counting, respectively. Vesicle density at the synaptic active zones was determined as the number of vesicles per μm², on an average area of 0.8 μm² per active zone. CCV density was determined within the limits of the presynaptic terminal on an average terminal area of 3.3 μm².

Squid Giant Synapse Preparation and Solutions.

All experiments were carried out at the Marine Biological Laboratory in Woods Hole, Mass. (MBL). As in previous research with this junction (Katz and Miledi 1967, 71, Llinas et al., 1976, 1981, Augustine and Charlton, 1986) one squid (Loligo paelli) stellate ganglion was rapidly removed from the mantle following decapitation and the stellate ganglion was dissected from the inner surface of the mantle under running seawater. Following isolation, the ganglion was placed in a recording chamber and submerged in artificial seawater (ASW). The ganglion was set in the chamber such that both the presynaptic and postsynaptic terminals could be directly visualized for microelectrode penetration. A total of 70 synapses were studied with the number of dissected preparation being close to one hundred fifty; some synapses dissected were not usable as clear anatomical and optimal transparency is required for experimental implementation stability.

RNS60.

RNS60 is a physically modified normal saline (0.9%) solution generated by using a rotor/stator device, which incorporates controlled turbulence and Taylor-Couette-Poiseuille (TCP) flow under high oxygen pressure (see Applicants U.S. Pat. Nos. 7,832,920; 7,919,534; 8,410,182; 8,445,546; 8,449,172; and 8,470,893, all incorporated herein by reference in their entireties for their teachings encompassing Applicants' device, methods for making the fluids, and the fluids per se). Briefly, for producing the RNS60 used in the working examples disclosed herein, sodium chloride (0.9%), USP pH 5.6 (4.5-7.0, Hospira), is processed using Applicants' patented device at 4° C. with a flow rate of 32 mL/s under 1 atm of oxygen backpressure (7.8 mL/s gas flow rate) while maintaining a rotor speed of 3,450 rpm. These conditions generate a strong shear layer at the interface between the vapor and liquid phases near the rotor cavities, which correlates with the generation of small bubbles from cavitation, shearing and other forces. The resulting fluid is immediately placed into glass bottles (KG-33 borosilicate glass, Kimble-Chase) and sealed using gray chlorobutyl rubber stoppers (USP class 6, West Pharmaceuticals) to maintain pressure and minimize leachables. When tested after 24 h, the oxygen content was 55±5 ppm (ambient temperature and pressure). Chemically, RNS60 contains water, sodium chloride, 50-60 parts/million oxygen, but no active pharmaceutical ingredients. The structure and activity of the fluids is stable for at least months or at least years at 4° C. in the closed containers.

Superfusion Solutions.

Two standard and one physically modified artificial seawater (ASW) solutions were used in these experiments. Salts were added to 1 liter of distilled water or a 40 ml bottle of physically modified water such that the final salt composition and pH were identical in every case (423 mM NaCl, KCl 8.27 mM, CaCl2 10 mM, MgCl2 50 mM, buffered to 7.2 with HEPES, salinity 3.121%). ASW made with distilled water or physically modified saline was prepared each day and keep at 4° until the start of the experiment. At the start of an experiment, the control ASW and one 40 ml bottle of RNS60 ASW was removed from the refrigerator, brought to room temperature, and the oxygen content measured. Several synapses (5-15) were dissected and studied each day. All experiments were carried out at room temperature (15-18° C.) as is our standard practice.

The physically modified saline was RNS60 ASW, made using RNS60 that contains oxygenated nanobubbles prepared with TCP flow. The standard ASWs were: 1) Control ASW, made using distilled H2O with air diffusion oxygenation (without bubbling); and 2) NS30612 ASW made using unprocessed normal saline from the same source solution as used to make RNS60. RNS60 and NS30612 were from Revalesio corporation, Tacoma, Wash. Removal of the synapse from the squid was carried out under running seawater. All procedures before beginning the recording sessions, the fine dissection and synapse impalement, were carried out using standard ASW because of the large volume of ASW required. In our initial experiments synaptic transmission in NS30612 was found to be indistinguishable from that recorded in our standard control ASW (not shown); ASW was used as the initial step in all experiments.

Oxygen Content Measurement.

Oxygen measurement of each superperfusate was determined using a Unisense MicroOptode near infrared (NIR, 760-790 nm) sensing probe (400 μm) corrected for temperature and salinity. The mean and s.e.m. of the oxygen content of each of the ASWs measured over 10 min were: 1) Control ASW 268±0.26 μmol/l (8.57 ppm) 2) RNS60 ASW 878±0.8 μmol/l (28.1 ppm); 3) Normal Saline (NS) 266±0.18 μmol/l (8.5 ppm). The oxygen content of RNS60 ASW is quite stable. Over the period of a typical experiment, about 30 min, oxygen content of the RNS60 ASW decreased by about 8,7%.

General Electrophysiology.

Following stable presynaptic and postsynaptic microelectrode impalement and the demonstration of synaptic transmission following presynaptic electrical stimulation the experimental procedure was initiated. The postsynaptic electrodes were beveled to reduce their resistance (<1 MΩ) and thus improved the signal/noise ratio. To evaluate changes in the RC properties of the postsynaptic membrane, the decay constant of the falling phase of the EPSPs was estimated using a built in curve fit function for a decaying exponential (exp Xoffset, Igor Pro, Wavemetrics, Inc).

Evoked Synaptic Transmission.

Single glass microelectrodes were inserted into the largest (most distal) presynaptic terminal and the corresponding postsynaptic axon. Evoked presynaptic and postsynaptic action potentials were recorded following our standard protocol (Llinas R. et al 1981). The synapse was activated either by extracellular electrical stimulation of the presynaptic axon via an insulated silver wire electrode pair or by direct depolarizing the presynaptic terminal through an intracellular electrode. Nerve stimulation was delivered as single stimulus or a train (250 ms at 200 Hz delivered at 1 Hz).

Spontaneous Release as Determined by Fourier Analysis of Postsynaptic Noise Level.

Spontaneous transmitter release was recorded postsynaptically as noise fluctuation of the postsynaptic membrane potential at the synaptic junction (Lin et al., 1990). Synaptic noise measurements provided a second method to assess synaptic viability, and a probe to understand possible effects of RNS60 on spontaneous synaptic vesicular release kinetics. By combining electrophysiological and ultrastructural analysis, we further assessed vesicular recycling properties on the synapse. This combination together with the use of mitochondrial inhibitors, such as oligomycin, allowed us to study the mechanism of RNS60 action on ATP synthesis (Lardy et al., 1958).

Synaptic noise was recorded using a Neurodata instrument amplifier (ER-91) with a Butterworth filter (0.1-1 kHz). Noise analysis was based on postsynaptic spontaneous unitary waveform determination via two exponential functions (Verveen and DeFelice, 1974), F(t) a[/[e−t/τd_e−t/τr] where a is an amplitude scaling factor and τd and τr are the decay and rise time constants respectively.

The power spectrum derived from the unitary potentials is S(f)=2na²(τd−τr)²/[1+4π²f²τ² d)(1+4π²f²τ²r)] where n is the rate of unitary release f and a, τd and τr are the same as above. The change in spontaneous release was quantified by averaging noise amplitude in noise frequencies between 20 and 200 Hz.

Noise Model.

In order to address the noise fluctuation changes observed following RNS60 based ASW we implemented a numerical solution for the noise profile (Lin et al., 1990). As in previous studies (Lin et al 1990), the time constant for the miniature potential rise time was determined as having a 0.2 ms and the fall time as 1.5 ms. The noise results following RNS60 were found to have a rise time of 0.2 and a fall time of 2.5 msec. The parameters for the RNS60 noise profile were selected by goodness of fit.

Voltage Clamp.

The voltage clamp experiments followed a standard protocol (Llinas et al. 1981). Briefly, two glass micropipette electrodes were inserted into the largest (most distal) presynaptic terminal digit at the synaptic junction site and a third micropipette impaled the postsynaptic axon at the junction site (Llinas R. et al 1981). One of the presynaptic electrodes was used for microinjection supporting the voltage clamp current feedback, while the second monitored membrane potential. Presynaptic voltage was measured using an FET input operational amplifier (Analog Devices model 515, Analog Devices, Inc., Norwood, Mass.). Current was injected by means of a high-speed, high-voltage amplifier (Burr-Brown Corp, 3584JM). Total current was measured by means of a virtual ground circuit (Teledyne Philbrick 1439, Teledyne Philbrick, Dedham, Mass.). The indifferent electrode consisted of a large silver-silver chloride plate located across the bottom of the chamber. To eliminate polarization artifacts, current was measured using an Ag—AgCl agar virtual ground electrode placed in the bath adjacent to the synapse. In most cases the time to plateau of the voltage microelectrode signal ranged from 50 to 150 μs.

ATP Synthesis.

ATP synthesis was determined using luciferin/luciferase light emitting measurements (McElroy W. D.1947). Luciferase was pressure-injected. into either the presynaptic or the postsynaptic terminal. Luciferin was added to the superfusate. Light emission was monitored and imaged using a single photon counting video camera (Argos-100 Hamamatsu Photonix). Light magnitude was determined using fifteen-second time integration periods. Oligomycin (0.25 mg/ml) was injected presynaptically using 50-100 ms pressure pulses and visualized directly using the photon counting camera. The volume injected was in the range of 0.5 to 1 pl, i.e., about 5 to 10% of the presynaptic terminal volume (Llinas R. et al. 1991) for a final concentration of 25.0 μg/ml, to block ATP synthesis.

Block of ATP Synthesis with Oligomycin.

Oligomycin (0.25 mg/ml) was injected presynaptically using 50-100 ms pressure pulses and visualized directly using the photon counting camera. The volume injected was in the range of 0.5 to 1 pl, i.e. about 5 to 10% of the presynaptic terminal volume (Llinas et al., 1991) for a final concentration of 25.0 μg/ml, to block ATP synthesis.

Ultrastructural Studies.

At the end of the electrophysiological recordings the stellate ganglion was immediately removed from the recording chamber and fixed by immersion in glutaraldehyde. Only synapses showing perfect preservation were accepted for analysis. Ultrastructural analysis was thus carried out on 240 active zones (AZ) from 8 synaptic terminals, as summarized in Table 1. The tissue was postfixed in osmium tetroxide, stained in block with uranium acetate, dehydrated and embedded in resin (Embed 812, EM Sciences). Ultrathin sections were collected on Pioloform (Ted Pella, Redding, Calif.) and carbon-coated single sloth grids, and contrasted with uranyl acetate and lead citrate. Morphometry and quantitative analysis of the synaptic vesicles were performed with the image J software (NIH, EUA). Electron micrographs were taken at an initial magnification of 20 or 30K. They were enlarged on a computer screen to a magnification of 50K for counting synaptic vesicles and to 75K for counting clathrin-coated vesicles (CCV). Synaptic vesicle density and the number of CCV at the synaptic active zones were determined as the number of vesicles per μm2.

Statistics; Morphology.

The synaptic vesicle density was analyzed by one-way ANOVA test (parametric test) followed by the Tukey test, and the CCV density was analyzed by the Mann-Whitney U test (non-parametric test). Both analyzes were realized in the Statistical Analysis System Software 10.0 (Statistical Analysis System Institute Inc., EUA). The data is presented as average±standard error). Electrophysiology. Analysis of the electrophysiological data was carried out in the SPSS environment (SPSS Statistics, IBM). Several measurements of each parameter were made for each experiment. Statistical analysis was carried out on the grand mean of the mean for each synapse. The t-test or independent samples ANOVA followed by the Tukey post-hoc test were used to determine significance. Three statistical thresholds are marked, P<0.05, P<0.01, P<0.001.

Database.

The data for this study were obtained from a total of 75 squid synapses yielding eighty-five experiments as summarized in Table 1. Synapses were included for analysis only if they had stable presynaptic and postsynaptic resting potentials and if the presynaptic and postsynaptic action potentials did not show signs of deterioration under control conditions.

TABLE 1
Summary of experiments comprising database for this study.

| | | | Control | *Oligomycin | |
|---|---|---|---|---|---|
| | | Control | PNS50 | Control | |
| Type of Experiment | Control | RNS60 | RNS60 | RNS60 | Total |
| Low oxygen content | — | 10 | — | — | 10 |
| Evoked release: | — | 5 | — | — | 5 |
| Single stimulus | | | | | |
| Evoked release: | 4 | 9 | 5 | 7 | 25 |
| Recuperation from | | | | | |
| repetitive stimulation | | | | | |
| Spontaneous Release | 5 | 6 | 5 | 9 | 25 |
| (noise and analysis) | | | | | |
| Presynaptic voltage | ref | 6 | — | — | 6 |
| clamp | | | | | |
| Intracellular ATP | — | 10 | — | — | 10 |
| generation | | | | | |
| (luciferin/luciferase) | | | | | |
| Total | 9 | 46 | 10 | 16 | 81 |

*Oligomycin was injected into the synapse.

References; incorporated herein by reference for their respective teachings in their entirety.
1. Lee V M, Goedert M, & Trojanowski J Q (2001) Neurodegenerative tauopathies. Annu Rev Neurosci 24:1121-1159.
2. Selkoe D J (2001) Alzheimer's disease: genes, proteins, and therapy. Physiol Rev 81(2):741-766.
3. Arancio O, et al. (2004) RAGE potentiates Abeta-induced perturbation of neuronal function in transgenic mice. EMBO J 23(20):4096-4105.
4. Moreno H, et al. (2009) Synaptic transmission block by presynaptic injection of oligomeric amyloid beta. Proc Natl Acad Sci USA 106(14):5901-5906.
5. LaFerla F M, Green K N, & Oddo S (2007) Intracellular amyloid-beta in Alzheimer's disease. Nat Rev Neurosci 8(7):499-509.
6. Yoshiyama Y, et al. (2007) Synapse loss and microglial activation precede tangles in a P301S tauopathy mouse model. Neuron 53(3):337-351.
7. Marx J (2007) Alzheimer's disease. A new take on tau. Science 316(5830):1416-1417.
Nixon R A, et al. (1992) The lysosomal system in neurons. Involvement at multiple stages of Alzheimer's disease pathogenesis. Ann NY Acad Sci 674:65-88.
9. Braak E, Braak H, & Mandelkow E M (1994) A sequence of cytoskeleton changes related to the formation of neurofibrillary tangles and neuropil threads. Acta Neuropathol 87(6):554-567.
10. Kowall N W & Kosik K S (1987) Axonal disruption and aberrant localization of tau protein characterize the neuropil pathology of Alzheimer's disease. Ann Neurol 22(5):639-643.
11. Perez M, Arrasate M, Montejo De Garcini E, Munoz V, & Avila J (2001) In vitro assembly of tau protein: mapping the regions involved in filament formation. Biochemistry 40(20):5983-5991.
12. Llinas R, et al. (1994) The inositol high-polyphosphate series blocks synaptic transmission by preventing vesiculat fusion: A squid giant synapse study. PNAS 91:12990-12993.
Lin J W, Sugimori M, Llinas R R, McGuinness T L, & Greengard P (1990) Effects of synapsin I and calcium/calmodulin-dependent protein kinase II on spontaneous neurotransmitter release in the squid giant synapse. Proc Natl Acad Sci USA 87(21):8257-8261.
14. Llinas R, McGuinness T L, Leonard C S, Sugimori M, & Greengard P (1985) Intraterminal injection of synapsin I or calcium/calmodulin-dependent protein kinase II alters neurotransmitter release at the squid giant synapse. Proc Natl Acad Sci USA 82(9):3035-3039.
15. Goedert M, Jakes R, & Vanmechelen E (1995) Monoclonal antibody AT8 recognises tau protein phosphorylated at both serine 202 and threonine 205. Neurosci Lett 189(3):167-169.
16. Micheva K D & Smith S J (2007) Array tomography: a new tool for imaging the molecular architecture and ultrastructure of neural circuits. Neuron 55(1):25-36.
17. Llinas R, Gruner J A, Sugimori M, McGuinness T L, & Greengard P (1991) Regulation by synapsin I and Ca(2+)-calmodulin-dependent protein kinase II of the transmitter release in squid giant synapse. J Physiol 436:257-282.
18. Jackson G R, et al. (2002) Humn wild-type tau interacts with wingless pathway components and produces neurofibrillary pathology in Drosophila. Neuron 34(4):509-519.
19. Avila J, Lucas J J, Perez M, & Hernandez F (2004) Role of tau protein in both physiological and pathological conditions. Physiol Rev 84(2):361-384.
20. Steinhilb M L, Dias-Santagata D, Fulga T A, Felch D L, & Feany M B (2007) Tau phosphorylation sites work in concert to promote neurotoxicity in vivo. Mol Biol Cell 18(12):5060-5068.
21. Hall G F, Yao J, & Lee G (1997) Human tau becomes phosphorylated and forms filamentous deposits when overexpressed in lamprey central neurons in situ. Proc Natl Acad Sci USA 94(9):4733-4738.
22. Lee S, Jung C, Lee G, & Hall G F (2009) Exonic point mutations of human tau enhance its toxicity and cause characteristic changes in neuronal morphology, tau distribution and tau phosphorylation in the lamprey cellular model of tauopathy. J Alzheimers Dis 16(1):99-111.
23. Mudher A, et al. (2004) GSK-3beta inhibition reverses axonal transport defects and behavioural phenotypes in Drosophila. (Translated from eng) Mol Psychiatry 9(5):522-530 (in eng).
24. Stamer K, Vogel R, Thies E, Mandelkow E, & Mandelkow E M (2002) Tau blocks traffic of organelles, neurofilaments, and APP vesicles in neurons and enhances oxidative stress. J Cell Biol 156(6): 1051-1063.
25. LaPointe N E, et al. (2009) The amino terminus of tau inhibits kinesin-dependent axonal transport: implications for filament toxicity. J Neurosci Res 87(2):440-451.
26. Yuan A, Kumar A, Peterhoff C, Duff K, & Nixon R A (2008) Axonal transport rates in vivo are unaffected by tau deletion or overexpression in mice. J Neurosci 28(7):1682-1687.
27. Diaz-Hernandez M, et al. (2010) Tissue-nonspecific alkaline phosphatase promotes the neurotoxicity effect of extracellular tau. J Biol Chem 285(42):32539-32548.
28. Gomez-Ramos A, Diaz-Hernandez M, Rubio A, Miras-Portugal M T, & Avila J (2008) Extracellular tau promotes intracellular calcium increase through M1 and M3 muscarinic receptors in neuronal cells. Mol Cell Neurosci 37(4):673-681.
29. Takahashi M, Amin N, Grant P, Pant H C. 1995 P13suc1 associates with a cdc2-like kinase in a multimeric cytoskeletal complex in squid axoplasm. J Neurosci 15(9):6222-9.
30. Morfini G, Szebenyi G, Elluru R, Ratner N, Brady S T. 2002 Glycogen synthase kinase 3 phosphorylates kinesin light chains and negatively regulates kinesin-based motility. EMBO J. 21(3):281-93.
31. Hanger D P, Seereeram A, Noble W. 2009 Mediators of tau phosphorylation in the pathogenesis of Alzheimer's disease. Expert Rev Neurother. (11):1647-66.
32. Roberson, E. D., Scearce-Levie, K., Palop, J. J., Yan, F., Cheng, I. H., Wu, T., Gerstein, H., Yu, G. Q., Mucke, L. (2007). Reducing endogenous tau ameliorates amyloid beta-induced deficits in an Alzheimer's disease mouse model. Science 316, 750-754.
33. Serulle Y., Morfini G., Pigino G., Moreira J. E., Sugimori M., Brady, S. T. and Llinas R. R (2007) 1-Methyl-4-phenylpyridinium induces synaptic dysfunction through a pathway involving caspase and PKC∂ enzymatic activities. PNAS 104:2437-2441.
34. Pigino G., Opalach K., Serulle., Moreira J. E., Sugimori M., Llinas, R. R., Brady S. T. (2007) 1-Methyl-4-phenylpyridinium affects fast axonal transport by activation of caspase and protein kinase C. PNAS 104:2442-2447.

### Example 2

### Intra-Axonal h-Tau41 Acutely Blocks Synaptic Transmission

Simultaneous presynaptic and post-synaptic axon impalements and the determination of normal synaptic transmission Winds R, et al., The inositol high-polyphosphate series blocks synaptic transmission by preventing vesiculat fusion: A squid giant synapse study. PNAS 91:12990-12993, 1994; Lin J W, et al., Effects of synapsin I and calcium/calmodulin-dependent protein kinase II on spontaneous neurotransmitter release in the squid giant synapse. Proc Natl Acad Sci USA 87(21):8257-8261, 1990; and Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985) was followed by presynaptic microinjection of human Tau 41 using a fluorescent dye/protein mix, that allowed direct visualization of presynaptic Tau injection. The preterminals, depending on impalement site and length reached a final concentration after diffusion of approximately 80 nM (see Materials and Methods).

FIG. 1 shows fluorescence imaging of presynaptic TAU 42 injection into the presynatic terminal. The time course for the diffusion of the protein is indicated by the diffusional speed of the fluorescence in the injected preterminal. The fluorescence image indicates that a time period of approximately 20 minutes was required for the injected protein to reach the distal presynaptic terminal furthest in the left. Magnification indicated by calibration bar in microns.

Presynaptic and postsynaptic potentials were recorded simultaneously under current-clamp configuration. Presynaptic spikes were activated every 5 min (low frequency protocol). With this paradigm, it was determined that 10-20 min after an injection of h-tau41 (depending on injection site in the preterminal), a reduction of transmitter release could be observed. With further time, a total block of transmission resulted, within 30 to depending on the length of the release zone in the preterminal axon (FIG. 2A, n=8). No modification of presynaptic spike amplitude or duration ensued (FIG. 2A). By contrast, following RNS60 based ASW superfusion, h-tau41-dependent transmitter block was prevented (FIG. 2 B).

Specifically, FIG. 2A shows an increase in latency and final block of postsynaptic spike generation and decrease in amplitude of the postsynaptic potential (lower arrows) at increasing time intervals following TAU injection. FIG. 2B shows that maintenance of synaptic transmission is present after 50 minutes following TAU injection (see fluorescence image insert to the right).

### Example 3

### Recovery of Spontaneous Neurotransmitter Release Following Reduction after TAU 41 Pre-Synaptic Injection was Observed

Beyond spike-initiated release, the possibility that h-tau41 may affect spontaneous transmitter release was directly evaluated using postsynaptic noise analysis. A detailed description of the technique has been published (Lin J W, et al., Effects of synapsin I and calcium/calmodulin-dependent protein kinase II on spontaneous neurotransmitter release in the squid giant synapse. Proc Natl Acad Sci USA 87(21):8257-8261, 1990; and Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985). In all preparations tested (n=6) the membrane noise recorded from the post-synaptic axon is followed by a noise level reduction that parallels the amplitude decrease of the evoked transmitted release. An example of the time course of noise level changes is illustrated in FIG. 3.

Specifically, FIG. 3 shows spontaneous synaptic noise reduction following TAU 41 injection. Control noise prior to TAU41 injection (purple dots). Following TAU 41 injection a marked reduction in noise is observed (green dots). Following RNS60 based ASW there is a recovery of noise level to above control 10 mins (blue) and 20 mins (green).

A good correlation was, in fact established between reduction of spontaneous release and the decrease in postsynaptic potential amplitude, indicating that these two forms of synaptic reduction have the same origin, that is a reduction in transmitter release, whether evoked or spontaneous.

Likewise the fact that both are reversed or prevented by superfusion with RNS60, indicates that TAU synaptic block can be prevented or reversed by RNS60 superfusion.

### Example 4

### RNS 60 ASW Reversed Synaptic Block without Modifying Calcium Currents

In agreement with previous results that transmission is rapidly blocked by h-tau41 (Moreno, et al 2011) without affecting the voltage dependent calcium current responsible for transmitter release (Llinas, et al 1981); confirming those findings, the present results indicate that reduction in transmitter release by TAU does not entail changes in presynaptic calcium currents (ICa²⁺). Rather, the amplitude and time course of ICa²⁺ (FIG. 4) as directly determined by presynaptic voltage clamp steps, after blocking voltage dependent K⁺ and Na⁺ currents (n=2), as previously described (Llinas R, et al., Intraterminal injection of synapsin I or calcium/calmodulin-dependent protein kinase II alters neurotransmitter release at the squid giant synapse. Proc Natl Acad Sci USA 82(9):3035-3039, 1985) indicate that ICa²⁺ amplitude and time course are not changed as determined at 5 min intervals over a period of 25 min following presynaptic injection of h-tau42, 80 nM.

FIG. 4A (upper left quadrant) shows a set of three superimposed voltage clamp results where presynaptic voltage steps (Pre V) that generated presynaptic inward calcium current (I Ca) and postsynaptic potentials (EPSP) are illustrated 10 minutes following presynaptic TAU 41 injection.

FIG. 4B (upper right quadrant), show a similar set of voltage clamp results 10 minutes following superfusion with RNS60 based ASW h-tau42 injection.

FIG. 4C (lower three sets of postsynaptic potentials) further shows before (green) and after (purple) presynaptic to show in more detail the increase on amplitude of the postsynaptic response to the presynaptic voltage steps. The middle traces are the before and after recordings of post synaptic potentials. In the center, is shown a direct comparison of these postsynaptic responses with the difference shown in yellow shading.

The results in FIG. 4 show, therefore, that a depression of transmitter release following TAU41 presynaptic injection, can be reversed by superfusion with RNS60 ASW.

### Example 5

### Intra-Axonal h-Tau42 Became Phosphorylated and Produced Synaptic Vesicle Aggregation

Since the aggregation of typical tau filaments is accompanied by the development of tau hyperphosphorylation, we investigated whether h-tau42 residues serine 202, threonine 205 and/or 231 were phosphorylated in the squid synapse. We used AT8 antibodies, as commonly used in neuropathological studies (Goedert, M., et al., Monoclonal antibody AT8 recognises tau protein phosphorylated at both serine 202 and threonine 205. Neurosci Lett 189(3):167-169, 1995). Here, we used immunohistochemistry in a variance of the array tomography technique (Micheva K D & Smith S J., Array tomography: a new tool for imaging the molecular architecture and ultrastructure of neural circuits. Neuron 55(1):25-36, 2007); we found that single sections (500 nM) allowed a clear view of the pre- and post-synaptic compartments (FIG. 5A). Anti-phospho-tau immunohistochemistry was detected as dot-like profiles in the presynaptic compartment in h-tau42 injected synapses (FIG. 5B; upper right panel). These were absent in synapses injected with vehicle (FIG. 5C; lower right panel). These findings confirm that h-tau42 becomes phosphorylated in the squid axon (Moreno et al 2011).

FIGS. 5A-5C show ultrastructural presynaptic changes secondary to h-tau42 injection. The structural changes that follow h-tau42 injection were addressed by rapidly fixing stellate ganglia (see Materials and Methods) after high or low-frequency stimulation protocols. The material consisted of injected synapses (62 synaptic active zones from 10 different squid) and vehicle injected synapses (control, 27 active zones in 5 synapses). The synapses were fixed ˜75-90 min after h-tau42 injection and processed for ultrastructural microscopy (see Materials and Methods).

As shown in representative control synapses (FIG. 5A), vesicles are normally present at the active zone, some in contact with the presynaptic terminal membrane (docked). By contrast, in h-tau42-injected synapses (FIG. 5 B) vesicles were often closely aggregated with electron dense material serving as a bonding matrix (red dot). Similar electron dense material was also observed around vesicles in contact with the active zone (red arrows). At a lower magnification (FIG. 5C) a large number of aggregated vesicular profiles are evident in the vicinity of the active (red dots). In RNS60 ASW superfussion, the synaptic morphology was quite similar to the vehicle-injected synapses (compare FIGS. 5A and 5C). Quantification of number of undocked, docked and clathrin-coated vesicles among the three groups (control, h-tau42 injected and h-tau42 injected and superfused with RNS60 ASW) was tabulated. There was a statistically significant reduction in the number of “docked vesicles” in h-tau42-injected synapses compared to axons injected with vehicle. This reduction was not seen in T817MA-treated squid following h-tau42 injection.

### Example 6

### Superfussion with RNS60 ASW Prevented Tau-Mediated Synaptic Block, Synaptic Vesicle Aggregation, and Decreased h-Tau42 Phosphorylation

RNS60 ASW physically modified water has been shown to ameliorate neuronal dysfunction. However, to study the potential effects of RNS60 on tau neuropathology, synapses were superfused with RNS60 as described in the methods. Electrophysiologically, as shown in FIG. 2B, no significant changes in the amplitude or time course of the pre- or post-synaptic potentials were observed. Further, ultrastructural studies in synapses used for the electrophysiological experiments demonstrated the number of docked vesicles recovered to the normal range in h-tau42/RNS60 superfused squid (31 active zones in 6 synapses) (10.0±0.6) compared to control synapses (10.1±0.7), with the presence of normal clathrin-coated vesicles (CCV) profiles (3.9±0.4). Also clear was a significant reduction, of electron dense vesicles clusters and electron dense active zones (FIGS. 4 and 5). It is thus concluded that superfusion with RNS60 ASW prevented the h-tau42 dependent synaptic vesicle clustering, indicating a close relation between such morphology and the synaptic transmitter release block observed electrophysiologically. Finally squid synapses superfused with RNS60 showed a significantly reduced signal of intra-axonal h-tau42 phosphorylation, as detected by AT8 immunohistochemistry (FIG. 5).

1. A method for treating pre-neuronal loss abnormalities in synaptic function, comprising administrating to a subject having neurons, an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies.

2. The method of claim 1, wherein preventing or reducing abnormalities in synaptic function that precede neuronal loss and/or NFTs formation comprises optimizing phosphorylation homeostasis in the neurons.

3. The method of claim 2, wherein optimizing phosphorylation homeostasis in the neurons comprises decreasing the phosphorylated/dephosphorylated ratio in proteins involved in synaptic vesicle function.

4. The method of claim 3, wherein decreasing the phosphorylated/dephosphorylated ratio in proteins involved in synaptic vesicle function comprises modulating tau-induced changes in the balance of kinases and phosphatases in the neurons.

5. The method of claim 3, wherein decreasing the phosphorylated/dephosphorylated ratio in proteins involved in synaptic vesicle function comprises decreasing the phosphorylated/dephosphorylated ratio of tau and/or synapsin 1.

6. The method of claim 5, comprising reducing tau hyperphosphorylation.

7. The method of claim 5, comprising reducing synapsin 1 phosphorylation.

8. The method of claim 1, wherein preventing or reducing abnormalities in synaptic function comprises modulating at least one presynaptic and/or postsynaptic response.

9. The method of claim 8, wherein modulating at least one presynaptic and/or postsynaptic response comprises an increase of spontaneous transmitter release.

10. The method of claim 8, wherein modulating at least one presynaptic and/or postsynaptic response comprises a modification of noise kinetics.

11. The method of claim 8, wherein modulating at least one presynaptic and/or postsynaptic response comprises an increase in a postsynaptic response.

12. The method of claim 11, comprising an increase in the postsynaptic response without an increase in presynaptic ICa⁺⁺ amplitude.

13. The method of claim 8, wherein modulating at least one presynaptic and/or postsynaptic response comprises a decrease in synaptic vesicle density and/or number at active zones.

14. The method of claim 13 further comprising an increase in the number of clathrin-coated vesicles, and large endosome like vesicles in the vicinity of the junctional sites.

15. The method of claim 8, wherein modulating at least one presynaptic and/or postsynaptic response comprises a marked increase in ATP synthesis leading to synaptic transmission optimization.

16. The method of claim 8, wherein modulating at least one presynaptic and/or postsynaptic response comprises an enhanced or more vigorous recovery of postsynaptic spike generation.

17. The method of claim 8, wherein modulating at least one presynaptic and/or postsynaptic response comprises increased ATP synthesis at the presynaptic and postsynaptic terminals.

18. A method for treating pre-neuronal loss abnormalities in synaptic function, comprising contacting neurons in vitro or ex vivo with an ionic aqueous solution comprising charge-stabilized oxygen-containing nanostructures having an average diameter of less than 100 nm in an amount and for a time period sufficient for preventing or reducing abnormalities in synaptic function that precede neuronal loss and/or NFTs formation in taupathies.

19. The method of claim 1, wherein preventing or reducing abnormalities in synaptic function that precede neuronal loss and/or NFTs formation comprises optimizing phosphorylation homeostasis in the neurons.

20. The method of claim 2, wherein optimizing phosphorylation homeostasis in the neurons comprises decreasing the phosphorylated/dephosphorylated ratio in proteins involved in synaptic vesicle function.

21. The method of claim 3, wherein decreasing the phosphorylated/dephosphorylated ratio in proteins involved in synaptic vesicle function comprises modulating tau-induced changes in the balance of kinases and phosphatases in the neurons.

22. The method of claim 3, wherein decreasing the phosphorylated/dephosphorylated ratio in proteins involved in synaptic vesicle function comprises decreasing the phosphorylated/dephosphorylated ratio of tau and/or synapsin 1.

23. The method of claim 5, comprising reducing tau hyperphosphorylation.

24. The method of claim 5, comprising reducing synapsin 1 phosphorylation.
